(19) United States
(12) Patent Application Publication
(10) Pub. No.: US 2020/0268989 A1
(43) Pub. Date: Aug. 27, 2020

(54) SYSTEM AND REGULATOR DEVICE FOR EVACUATING SMOKE FROM A LAPAROSCOPIC FIELD AND METHOD OF EVACUATING SMOKE FROM A LAPAROSCOPIC FIELD

(71) Applicant: Boehringer Technologies, LP, Phoenixville, PA (US)

(72) Inventors: Christopher L. Radl, Malvern, PA (US); Trevor Smith, West Chester, PA (US); Michael Reed Vennel, Phoenixville, PA (US); William Charles Dackis, Philadelphia, PA (US)

(21) Appl. No.: 16/792,433

(22) Filed: Feb. 17, 2020

Related U.S. Application Data
(60) Provisional application No. 62/811,027, filed on Feb. 27, 2019.

(51) Int. Cl. A61M 13/00; A61M 39/24; A61B 17/34

(52) U.S. Cl. CPC A61M 13/003; A61M 39/24; A61B 17/3474; A61M 2039/229; A61M 2205/3337; A61B 2217/005; A61M 2205/3344

(57) ABSTRACT

A smoke evacuator system includes a regulator device configured for coupling to a vacuum source for evacuating smoke from a laparoscopic field within the body of a patient. The regulator device is pneumatic-operated and configured to continuously monitor pressure of insufflation gas within the laparoscopic field and to automatically apply suction from the vacuum source to the laparoscopic field when the pressure monitored reaches a preset level, whereupon smoke within the laparoscopic field is evacuated from the laparoscopic field. In one embodiment the smoke is evacuated via a conventional trocar. In another embodiment, where the laparoscopic field is confined, the smoke is evacuated by means of a smoke probe.

### CROSS-REFERENCE TO RELATED APPLICATIONS

This utility application claims the benefit under 35 U.S.C. § 119(e) of Provisional Application Ser. No. 62/811,027 filed on Feb. 27, 2019, entitled System And Regulator Device For Evacuating Smoke From A Laparoscopic Field And Method Of Evacuating Smoke From A Laparoscopic Field. The entire disclosure of this provisional application is incorporated by reference herein.

### FIELD OF THE INVENTION

This invention relates generally to medical devices and methods and more particularly to devices and methods for removing smoke from a laparoscopic surgical field.

### SPECIFICATION

### Background of the Invention

During a laparoscopic surgical procedure it is common for smoke to be created within the interior space in which the procedure is carried out (i.e., the “laparoscopic field” or “laparoscopic space”). The smoke can be created in various ways, e.g., by cauterization, laser incision, coagulation, vaporization, etc. In any case the smoke created can obscure the laparoscopic field for the surgeon, thereby making the laparoscopic procedure more difficult.

One technique for clearing the laparoscopic field of smoke is to use a trocar or other instrument extending into the field so that the positive pressure within the field will force the smoke out of the trocar and into a filter where the smoke will be collected. One such device using this passive smoke removal technique is the SeeClear® Surgical Smoke Evacuation System available from CooperSurgical, Inc. However, that passive smoke removal technique is not particularly effective due to the low positive pressure within the laparoscopic field, thereby rendering the smoke evacuation process somewhat slow and not as effective as possible. There are other devices currently available to actively withdraw the smoke from the laparoscopic field using a vacuum or suction. One such device is the Laparoshield Laparoscopic Smoke Filtration System sold by Pall Corporation. It consists of a device which is configured to be connected between a trocar or other device extending into the insufflated abdomen and to the hospital's vacuum or suction line. The device includes a manually operable button which the surgeon can press to couple the suction from hospital's suction line through the trocar to the laparoscopic field, whereupon the suction applied will draw the smoke out of the field. Since the level of suction provided by the hospital's suction line is significantly higher than the amount of suction required to clear smoke from the laparoscopic field, that device includes a bleed port in communication with the ambient air to reduce the level of suction applied when the button is pressed, otherwise the normal level of suction produced at the hospital's suction line would rapidly collapse the insufflated abdomen. Another smoke evacuation device utilizing the hospital's suction line is the PlumePort® ActiV® Laparoscopic Smoke Filtration Device available from Buffalo Filter, LLC. Still another smoke evacuation device utilizing the hospital's suction line is the PneuVIEW®XE smoke elimination system available from LEXION Medical, LLC. Coviden, AG provides a complex self-contained system, called the RapidVac™ Smoke Evacuator System, which is arranged for use with an electrosurgical generator to evacuate electrosurgical smoke and laser plume from a laparoscopic field.

While the above prior art may be generally suitable for the intended purpose of clearing smoke from a laparoscopic field, they each exhibit one or more of the following drawbacks, cost, complexity, effectiveness, efficiency and ease of use.

Thus, a need exists for a smoke evacuation system including a device which is low in cost, easy to use, automatic in operation, and effective for clearing smoke from a laparoscopic field. The subject invention addresses that need by providing a completely pneumatic, low cost (e.g., disposable) smoke evacuator regulator device which is configured for automatically and continuously removing smoke from a laparoscopic field.

### BRIEF SUMMARY OF THE INVENTION

One aspect of this invention is a smoke evacuator regulator device configured for coupling to a vacuum source for evacuating smoke from a laparoscopic field within the body of a patient, the laparoscopic field being insufflated with insufflation gas under positive pressure. The smoke evacuator regulator device is pneumatically-operated and configured to continuously monitor pressure of the gas within the laparoscopic field and to automatically apply suction from the vacuum source to the laparoscopic field when the pressure monitored reaches a set-point, whereupon smoke within the laparoscopic field is evacuated from the laparoscopic field.

In accordance with one preferred aspect of the smoke evacuator regulator device, it comprises a housing, a first device port, a second device port, a third device port and a valve. The first device port is located in the housing and configured for coupling to the laparoscopic field for monitoring the pressure of the gas in the laparoscopic field. The second device port is located in the housing and configured for coupling to the laparoscopic field for evacuating smoke from the laparoscopic field via the second device port. The third device port is located in the housing and configured for coupling to the vacuum source. The valve is located in the housing, is in a normally closed state and is coupled between the second device port and the third device port. The valve is operative in automatic response to the pressure of the gas monitored at the first device port, whereupon the valve opens to an open state to enable suction from the vacuum source to be applied through the third device port to the second device port when the pressure monitored at the first device port reaches the set-point, whereupon smoke within the laparoscopic field is evacuated from the laparoscopic field via the second device port and the third device port.

In accordance with another preferred aspect of the smoke evacuator regulator device of this invention, it additionally comprises a pressure chamber and a movable diaphragm. The pressure chamber is in fluid communication with the first port. The movable diaphragm forms a portion of the pressure chamber and is coupled to the valve. The movable diaphragm is biased to apply a bias force in opposition to pressure within the pressure chamber. The bias force establishes the set-point.

In accordance with another preferred aspect of the smoke evacuator regulator device of this invention, the bias force is adjustable.

In accordance with another preferred aspect of the smoke evacuator regulator device of this invention, the bias force is established by a spring.

In accordance with another preferred aspect of the smoke evacuator regulator device of this invention, the smoke evacuator regulator device comprises a housing, a diaphragm, a rotatable dial, an engagement member, a spring and a valve. The housing includes a pressure monitoring chamber, a first port, a second port, a third port. The pressure monitoring chamber is configured for fluid communication with the laparoscopic field via the first port, whereupon some of the insufflation gas is within the pressure monitoring chamber. The second port is configured for coupling to the laparoscopic field for evacuating smoke from the laparoscopic field via the second port. The third port is configured for coupling to the vacuum source. The diaphragm establishes a wall of the pressure monitoring chamber and is movable in response to a force applied thereto by the pressure of the insufflation gas within the pressure monitoring chamber. The rotatable dial is coupled to the housing and rotatable through an arc about a rotation axis between a first angular position and a second angular position, and vice versa, to establish an operating range for the smoke evacuator device. The engagement member is coupled to the rotatable dial and configured to cooperate with a stop member to adjust the operating range up or down to a desired operating range. The spring is coupled to the rotatable dial and configured to apply a bias force to the diaphragm in opposition to the force applied by the pressure of the insufflation gas within the pressure monitoring chamber. The bias force is adjustable within the desired operating range in response to rotation of the dial about the axis between the first angular position and the second angular position to establish the set-point. The set-point is adjustable within the desired operating range. The valve comprises a movable valve member and a valve seat in the housing. The valve is normally in a closed state isolating the second port from the third port. The movable valve member is connected to the movable diaphragm and movable therewith in automatic response to the pressure of the insufflation gas in the pressure monitoring chamber, whereupon the valve opens to an open state to enable suction from the vacuum source to be applied through the third port to the second port when the gas pressure monitored by the pressure monitoring chamber reaches the set-point, whereupon smoke within the laparoscopic field is evacuated from the laparoscopic field via the second port and the third port.

In accordance with another preferred aspect of the smoke evacuator regulator device of this invention, the stop member is located within the housing. The rotatable dial includes plural spaced apart openings extending in an arc about the rotation axis. The engagement member is configured to be located in any of one the openings to establish the desired operating range.

In accordance with another preferred aspect of the smoke evacuator regulator device of this invention, the stop member includes a first surface and a second surface, and wherein the engagement member is configured to engage the first surface at the first angular position and to engage the second surface at the second angular position.

In accordance with another preferred aspect of the smoke evacuator regulator device of this invention, the smoke evacuator regulator device additionally comprises a control pressure chamber within the housing configured to be at atmospheric pressure and defined between the rotatable dial and the diaphragm. The rotatable dial is configured to move toward the diaphragm by the rotation of the rotatable dial in a first rotational direction about the rotational axis and to move away from the diaphragm by the rotation of the dial in a second and opposite rotation direction. The spring is interposed between the rotatable dial and the diaphragm in the control pressure chamber, whereupon the bias force provided by the spring is increased upon rotation of the dial in the first rotational direction and the bias force provided by the spring is decreased upon rotation of the dial is the second and opposite rotational direction.

In accordance with another preferred aspect of the smoke evacuator regulator device of this invention, the housing comprises a base and a cap. The diaphragm is interposed between the base and the cap. The spring is interposed between the rotatable dial and the diaphragm. The rotatable dial is threadedly secured to the cap.

In accordance with another preferred aspect of the smoke evacuator regulator device of this invention, the spring comprises a helical compression spring having a longitudinal axis. The spring is interposed between the rotatable dial and a portion of the diaphragm, with the longitudinal axis of the spring being coaxial with the rotation axis.

In accordance with another preferred aspect of the smoke evacuator regulator device of this invention, the smoke evacuator regulator device additionally comprises a rotatable isolation disk interposed between the rotatable dial and the spring, whereupon rotation of the rotatable dial in either the first or second direction about the rotation axis does not cause the spring to rotate about the rotation axis.

In accordance with another preferred aspect of the smoke evacuator regulator device of this invention, the valve seat is formed of a resilient material. The movable valve member includes an end surface configured to engage the valve seat when the valve is in the normally closed state, and to be disengaged from the valve seat when the valve is in the open state.

In accordance with another preferred aspect of the smoke evacuator regulator device of this invention, the rotatable dial includes a detent mechanism for holding the rotatable dial at any rotational position establishing the set-point.

In accordance with another preferred aspect of the smoke evacuator regulator device of this invention, the detent mechanism comprises plurality of radially extending fins configured to be engaged by the engagement member to hold the rotatable dial at any rotational position establishing the set-point.

In accordance with another preferred aspect of the smoke evacuator regulator device of this invention, the smoke evacuator regulator device forms a portion of a system comprising a first tube, a second tube and a third tube. The first tube is configured to be connected to a first trocar extending into the laparoscopic field. The second tube is configured to be connected to a second trocar extending into the laparoscopic field. The third tube is configured to be connected to a canister which is connected to the vacuum source.

Another aspect of this invention is a system for evacuating smoke from a laparoscopic field within the body of a patient, the laparoscopic field being insufflated with insufflation gas under positive pressure. The system comprises a first instrument port in fluid communication with the laparoscopic field for monitoring the pressure of the gas in the laparoscopic field, a second instrument port in fluid communication with the laparoscopic field for evacuating smoke from the laparoscopic field via the second instrument port, and a smoke evacuator regulator device. The smoke evacuator regulator device comprises a housing, a first device port, a second device port, a third device port and a valve. The first device port is located in the housing and configured for coupling to the first instrument port for monitoring the pressure of the gas in the laparoscopic field. The second device port is located in the housing and configured for coupling to the second instrument port for evacuating smoke from the laparoscopic field via the second device port. The third device port is located in the housing and configured for coupling to the vacuum source. The valve is located in the housing and is in a normally closed state. The valve is coupled between the second device port and the third device port. The valve is operative in automatic response to the pressure of the gas monitored at the first device port, whereupon the valve opens to an open state to enable suction from the vacuum source to be applied through the third device port to the second device port when the pressure monitored at the first device port reaches a set-point, whereupon smoke within the laparoscopic field is evacuated from the laparoscopic field via the second device port and the third device port.

In accordance with one preferred aspect of the system of this invention, the smoke evacuator regulator device is pneumatically-operated and additionally comprises a pressure chamber and a movable diaphragm. The pressure chamber is in fluid communication with the first device port. The movable diaphragm forms a portion of the pressure chamber and is coupled to the valve. The movable diaphragm is biased to apply a bias force in opposition to pressure within the pressure chamber. The bias force establishes the set-point.

In accordance with another preferred aspect of the system of this invention, the bias force is adjustable.

In accordance with another preferred aspect of the system of this invention, the bias force is established by a spring.

In accordance with another preferred aspect of the system of this invention, the first instrument port forms a portion of a first instrument extending into the laparoscopic field, and wherein the second instrument port forms a portion of a second instrument extending into the laparoscopic field.

In accordance with another preferred aspect of the system of this invention, the first instrument is a trocar and wherein the second instrument is a trocar.

In accordance with another preferred aspect of the system of this invention, the laparoscopic field is confined, and wherein the system comprises a gel port and a smoke evacuating probe. The gel port includes the first instrument port, and another instrument port configured to be coupled to an insufflator supplying the insufflation gas under positive pressure to the laparoscopic field via the other instrument port. The gel port includes a penetrable portion. The second instrument port comprises a portion of the smoke evacuating probe. The smoke evacuating probe comprises an elongated needle and a one-way luer stop cock. The elongated needle has a longitudinal passageway extending therethrough. The one-way luer stop cock includes a luer connector and a movable lever. The luer connector is configured to be brought into fluid communication with the longitudinal passageway when the movable lever is moved to a predetermined position. The elongated needle is configured for penetrating the penetrable portion of the gel port.

Another aspect of this invention is a method of evacuating smoke from a laparoscopic field within the body of a patient, the field being insufflated with gas under positive pressure. The method comprises continuously monitoring pressure of the gas within the laparoscopic field via a first instrument port in fluid communication with the laparoscopic field. A pneumatically-operated smoke evacuator regulator device is coupled to a vacuum source and to a second instrument port in fluid communication with the laparoscopic field. Suction is automatically applied from the vacuum source to the laparoscopic field when the pressure monitored reaches a set-point, whereupon smoke within the laparoscopic field is evacuated from the laparoscopic field via the second instrument port and the vacuum source.

In accordance with one preferred aspect of the method of this invention, the pneumatically-operated smoke evacuator regulator device continuously monitors the pressure of the insufflation gas in the laparoscopic field via the first instrument port and the method additionally comprises coupling the pneumatically-operated smoke evacuator regulator device to a second instrument port in fluid communication with the laparoscopic field. A set-point for a desired pressure of the insufflation gas within the laparoscopic field is established. The pneumatically-operated smoke evacuator regulator device is operated to monitor the pressure of the insufflation gas in the laparoscopic field and automatically couples the second instrument port to the vacuum source when the pressure of the gas monitored in the laparoscopic field reaches the set-point, whereupon smoke within the laparoscopic field is evacuated from the laparoscopic field via the second instrument port and the vacuum source.

In accordance with another preferred aspect of the method of this invention, the laparoscopic field is confined and wherein the method additionally comprises providing a smoke probe including the second instrument port. The smoke probe is extended into the confined laparoscopic field. The smoke probe additionally comprises a valve and a thin elongated tubular member having a longitudinally extending passageway extending therethrough and terminating at an open distal end. The valve is interposed between the second instrument port and the longitudinally extending passageway. The open distal end of the smoke probe is disposed closely adjacent a source of smoke within the confined laparoscopic field. The valve is opened, whereupon smoke produced by the source of smoke is evacuated from the confined laparoscopic field via the smoke probe.

In accordance with another preferred aspect of the method of this invention, the first instrument port comprises a portion of a gel port configured for location within an opening in the body of the patient in communication with the laparoscopic field. The gel port includes a penetrable member, and wherein the method additionally comprises penetrating the penetrable member by the thin elongated tubular member of the smoke probe.

### BRIEF DESCRIPTION OF SEVERAL VIEWS OF THE DRAWINGS

FIG. 1A is an illustration of one exemplary regulator device constructed in accordance with this invention shown in use as part of an exemplary system to evacuate smoke from a laparoscopic field, e.g., an insufflated abdomen of a patient;

FIG. 1B is an illustration, similar to FIG. 1A, but showing the exemplary regulator device of FIG. 1A as part of another exemplary system including a smoke probe constructed in accordance with this invention to evacuate smoke from a confined laparoscopic field of a patient;

FIG. 2 is an enlarged isometric view of the regulator device shown in FIG. 1 taken from above;

FIG. 3 is an enlarged isometric view of the regulator device shown in FIG. 1 taken from below;

FIG. 4 is an exploded isometric view of the components making up regulator device shown in FIGS. 1-3;

FIG. 5 is an isometric view of one component, i.e., a rotatable dial, of the regulator device shown in FIGS. 1-3;

FIG. 6 is an isometric view of the other components, i.e., a set screw, a spring base, a spring, a cap, a diaphragm and a body or base, making up the regulator device shown in FIGS. 1-3;

FIG. 7 is an isometric view of the cap, the diaphragm, the body or base, and a string of the regulator device shown in FIGS. 1-3;

FIG. 8 is an enlarged isometric view, partially in section, of the diaphragm and a center stiffener of the regulator device shown in FIGS. 1-3;

FIG. 9 is an isometric view of the cap, the body or base, and the string of the regulator device shown in FIGS. 1-3;

FIG. 10 is an isometric view of the body or base of the regulator device shown in FIGS. 1-3;

FIG. 11 is an isometric view of the string, a valve member or piston, and a tubular seal forming a valve seat of the regulator device shown in FIGS. 1-3;

FIG. 12 is an enlarged isometric view in vertical section of the assembled regulator device shown in FIGS. 1-3;

FIG. 13 is another enlarged isometric view in vertical section of the assembled regulator device shown in FIGS. 1-3; and

FIG. 14 is an enlarged plan view of the smoke probe shown in FIG. 1B;

FIG. 15 is an isometric view in longitudinal section of the smoke probe shown in FIG. 14;

FIG. 16 is an illustration of one exemplary smoke evacuator device constructed in accordance with this invention shown in use as part of an exemplary system to evacuate smoke from a laparoscopic field, e.g., an insufflated abdomen of a patient;

FIG. 17 is an enlarged isometric view of the evacuator device shown in FIG. 16 taken from above, with the dial of the device being in its down or closed position;

FIG. 18 is an enlarged isometric view of the evacuator device shown in FIG. 17 taken from below;

FIG. 19 is an exploded isometric view of the components making up the evacuator device shown in FIGS. 16-18;

FIG. 20A is an isometric view of one component, i.e., a rotatable dial, of the evacuator device shown in FIGS. 16-18;

FIG. 20B is a cross-sectional view isometric view of the rotatable dial shown in FIG. 20A;

FIG. 21A is an isometric view taken from above of another component, i.e., a cap member, making up a portion of a housing assembly of the evacuator device shown in FIGS. 16-18;

FIG. 21B is an isometric view taken from below of the cap member shown in FIG. 21A;

FIG. 22A is an isometric view taken from above of another component, i.e., a valve member, making up the evacuator device shown in FIGS. 16-18;

FIG. 22B is an isometric view taken from below of the valve member shown in FIG. 22A;

FIG. 22C is a cross-sectional view isometric view of the valve member shown in FIGS. 22A and 22B;

FIG. 23A is an isometric view taken from above of another component, i.e., a diaphragm, making up the evacuator device shown in FIGS. 16-18;

FIG. 23B is an isometric view taken from below of the diaphragm shown in FIG. 23A;

FIG. 23C is a side elevation view taken from below of the diaphragm shown in FIG. 23A;

FIG. 24A is an isometric view taken from above of another component, i.e., a base or body member, making up another portion of the housing assembly of the evacuator device shown in FIGS. 16-18;

FIG. 24B is an isometric view taken from below of the base or body member shown in FIG. 24A;

FIG. 25A is an isometric view taken from above of another component, i.e., an adapter, making up the evacuator device shown in FIGS. 16-18;

FIG. 25B is a cross-sectional view isometric view of the adapter shown in FIG. 25A;

FIG. 26A is an isometric view taken from above of another component, i.e., a spring base, making up the evacuator device shown in FIGS. 16-18;

FIG. 26B is a cross-sectional view isometric view of the spring base shown in FIG. 26A;

FIG. 27 is an isometric view taken from the side of another component, i.e., a helical compression spring, making up the evacuator device shown in FIGS. 16-18;

FIG. 28 is an isometric view taken from the side of another component, i.e., an engagement member (e.g., a set-screw), making up the evacuator device shown in FIGS. 16-18;

FIG. 29A is an isometric view taken from the side of another component, i.e., a valve seat (e.g., a square profile O-ring), making up the evacuator device shown in FIGS. 16-18;

FIG. 29B is a cross-sectional view isometric view of the valve seat shown in FIG. 29A;

FIG. 30A is a vertical cross-sectional view of the evacuator device shown in FIGS. 16-18 taken through a vertical plane extending through a smoke evacuation port of the device;

FIG. 30B is an isometric cross-sectional view of the evacuator device shown in FIG. 30A, but taken through a vertical plane perpendicular to the cross-section plane of FIG. 30A;

FIG. 30C is an isometric cross-sectional view of the evacuator device shown in FIG. 30A, taken through a similar vertical plane to vertical plane of the cross-section of FIG. 30A, with the dial and engagement member of the evacuator device removed;

FIG. 30D is an isometric cross-sectional view of the evacuator device shown in FIG. 30A, taken through a vertical plane parallel to vertical plane of the isometric cross-section of FIG. 30C to extend through a portion of a pressure monitoring port of the evacuator device and with the dial and engagement member of the evacuator device removed;

FIG. 31 is an enlarged isometric view of the evacuator device shown in FIG. 16 taken from above, with the dial of the evacuator device being in its up or open position;

FIG. 32 is an enlarged vertical cross-sectional view of the evacuator device shown in FIG. 31 taken through a vertical plane extending through the smoke evacuation port of the evacuator device;

FIG. 33 is an isometric view of another component, a conventional sheet or drape clip, making up another component of the evacuator device shown in FIGS. 16-18;

FIG. 34 is a top view of the dial shown in FIGS. 20A-20B, with the engagement member (e.g., set-screw) located at an intermediate position in a group of adjustment holes to establish a mid-range of set-points for the operation of the evacuator device shown in FIGS. 1-3;

FIG. 35A is a bottom view of the cap shown in FIGS. 21A-21B with the engagement member (e.g., set-screw) located at the left-most position in the adjustment holes (relative to a top view of the dial as shown in FIG. 34) to establish the lowest range of set-points for the operation of the evacuator device shown in FIGS. 16-18;

FIG. 35B is a bottom view of the cap shown in FIGS. 21A-21B with the engagement member (e.g., set-screw) located at the intermediate position in the adjustment holes like shown in FIG. 32 to establish the mid-range of set-points for the operation of the evacuator device shown in FIGS. 16-18; and

FIG. 35C is a bottom view of the cap shown in FIGS. 21A-21B with the engagement member, e.g., set-screw) located at the right-most position in the adjustment holes (relative to a top view of the dial as shown in FIG. 34) to establish the highest range of set-points for the operation of the evacuator device shown in FIGS. 16-18.

### DETAILED DESCRIPTION OF THE PREFERRED EMBODIMENTS OF THE INVENTION

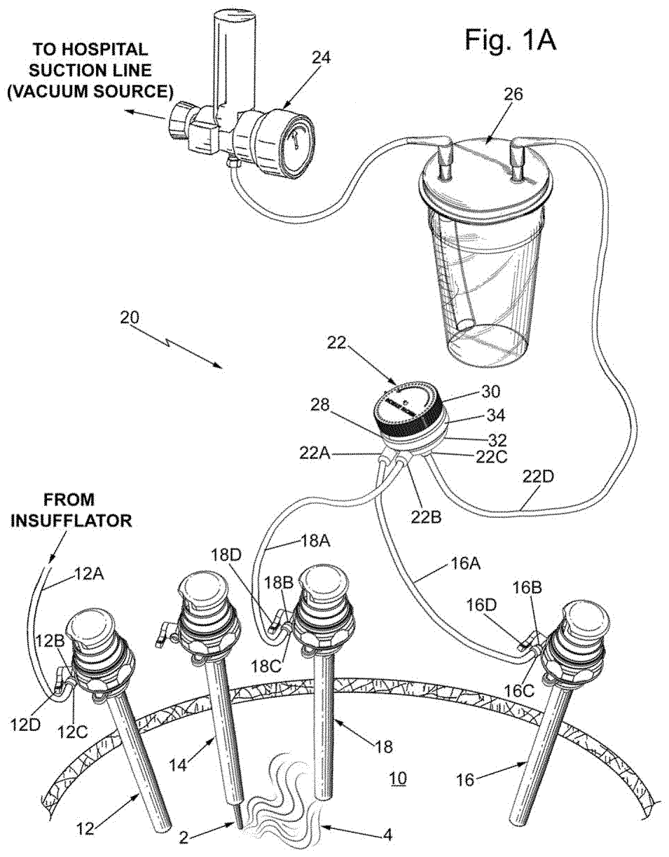

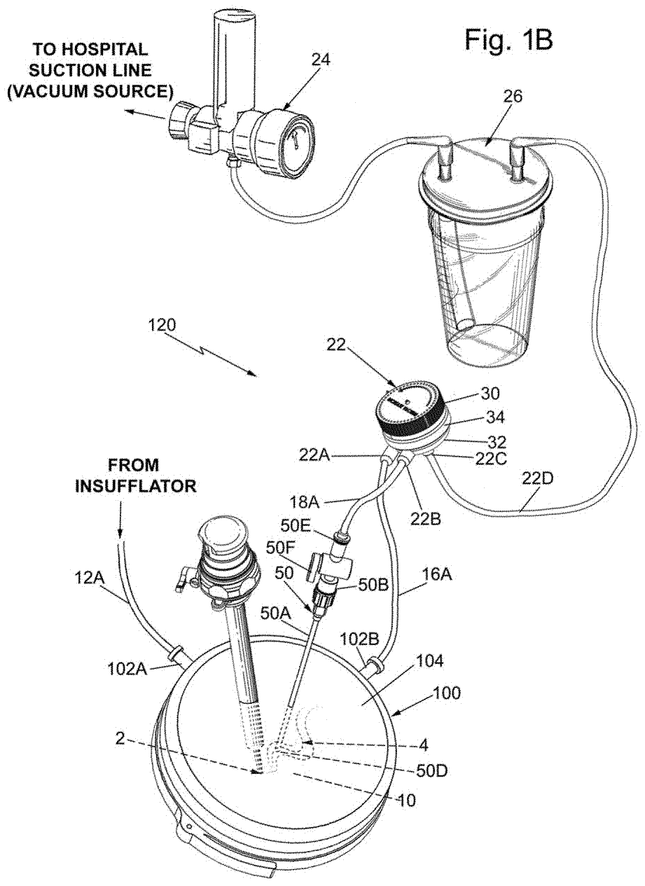

Referring now to the various figures of the drawing wherein like reference characters refer to like parts, there is shown in FIG. 1A one exemplary system 20 for evacuating smoke from a laparoscopic field within the body of a patient. In this case the laparoscopic field constitutes an abdomen 10 of a living being which has been insufflated with a gas under positive pressure by means of a trocar 12 extending into the abdomen. The trocar 12 is conventional device that includes a female luer connector 12B. The luer connector 12B is connected to one end of a flexible tube 12B by use of conventional male barbed connector 12C. An on-off valve (not shown) is located in the trocar and is coupled to a pivotable lever 18D to either open or close the luer connector, depending upon the rotational position of the lever. The opposite end of the flexible tube 12A is connected to a conventional insufflator (not shown). Accordingly gas under any conventional positive pressure used to insufflate a laparoscopic field (e.g., 15 mm Hg) can be delivered from insufflator into the field 10 via the trocar 12. Another trocar 14 is shown extending into abdomen of the patient. In this exemplary system the trocar 14 serves as the means through which a laparoscopic instrument 2 is extended to perform some laparoscopic procedure in the insufflated abdomen. The instrument 2 can be any type of instrument used in laparoscopic procedure. The use of some such instruments, e.g., a cauterizer, results in the creation of smoke 4 at the instrument. That smoke, if not evacuated from the laparoscopic field can obscure the vision of the surgeon carrying out the procedure and thus should be evacuated.

The system 20 includes a smoke evacuator regulator device 22 constructed in accordance with this invention. The smoke evacuator regulator device 22 may also be referred to herein as a regulator device or a smoke evacuator device. In any case it is configured for coupling to a vacuum source, e.g., a hospital's suction line, to continuously evacuate the smoke 4 from the laparoscopic field. The regulator device 22 will be described in detail shortly. Suffice it for now to state that the regulator device includes three device ports, namely, a first device port 22A, a second device port 22B, and a third device port 22C. The first device port 22A is coupled to another trocar 16 via a flexible tube 16A. The trocar 16 is also a conventional device that includes a female luer connector 16B. The luer connector 16B is connected to one end of the flexible tube 16A by use of conventional male barbed connector 16C. An on-off valve (not shown) is located in the trocar 16 and is coupled to a pivotable lever 16D to either open or close the luer connector 16B, depending upon the rotational position of the lever. The trocar 16 extends into the laparoscopic field, e.g., the insufflated abdomen, to monitor the pressure of the gas therein. The second device port 22B is coupled to still another trocar 18 via a flexible tube 18A. The trocar 18 is also a conventional device that includes a female luer connector 18B. The luer connector 18B is connected to one end of the flexible tube 18A by use of conventional male barbed connector 18C. An on-off valve (not shown) is located in the trocar 18 and is coupled to a pivotable lever 18D to either open or close the luer connector 18B, depending upon the rotational position of the lever. The trocar 18 extends into the insufflated abdomen to serve as the means for evacuating the smoke 4 from the laparoscopic field. The third device port 22C is coupled to the vacuum source, e.g., the hospital's suction line, via a flexible tube 22D. In the exemplary system 20 shown in FIG. 1A a conventional suction regulator 24 and a conventional canister 26 are connected in series between the tube 22D and the vacuum source. The suction regulator and the canister are optional, and one or both need not be used, e.g., the tube 22D from the smoke evacuator regulator 22 can be directly connected to the vacuum source, if desired.

As will be described in detail later the regulator device 22, when connected in the system 20 like shown in FIG. 1A, operates to continuously monitor pressure of the gas within the laparoscopic field 10 via the trocar 16 and automatically applies suction from the vacuum source to the laparoscopic field via the trocar 18 when the pressure monitored reaches a preset level, whereupon smoke 4 within the laparoscopic field is evacuated from the laparoscopic field through the trocar 18, its communicating tube 18A, and the device 22 to the vacuum source.

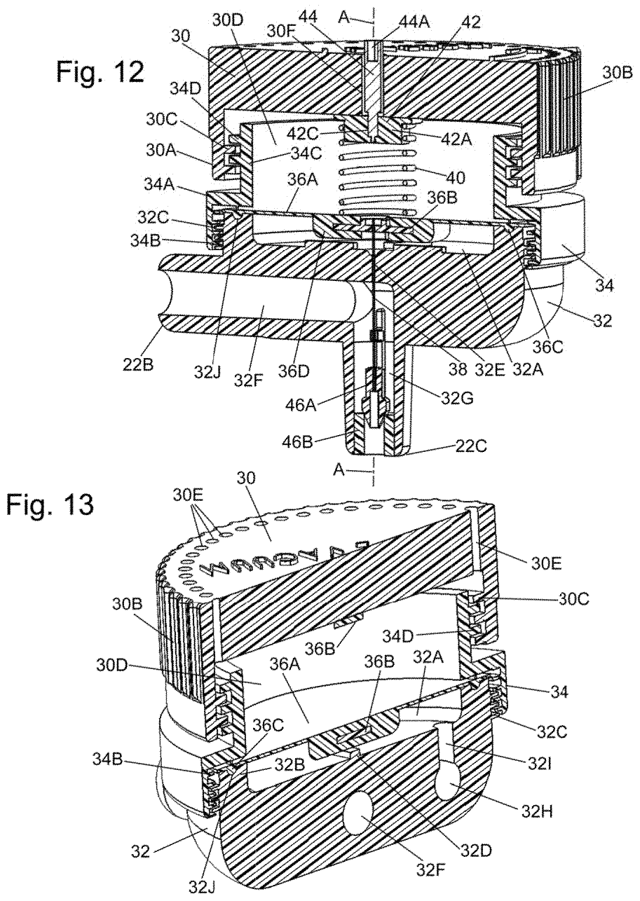

The regulator device 22 basically comprises a housing 28, a dial 30 and a valve assembly (to be described later). The housing 28 includes a body or base 32, a cap 34, a diaphragm assembly 36, a string 38, a spring 40, a spring base 42, a set screw 44, and a valve assembly 46. The body or base 32 is best seen in FIGS. 10, 12 and 13 is a cup shaped member formed of any suitable rigid material, e.g., Acrylonitrile Butadiene Styrene (“ABS”). The body or base includes a recess forming a pressure chamber or cavity 32A bounded by an annular wall 32B. The annular wall includes a helical external thread 32C. The bottom surface of the chamber or cavity 32A includes four low height linear stand-offs 32D extending perpendicularly to one another in a cruciform configuration. A very small diameter central aperture 32E is located centered in the recess between the inner ends of the stand-offs. The aperture 32E is in communication with a radially extending passageway 32F in the body or base 32. The passageway 32F terminates at its outer end in a tubular section which forms the port 22B. The body or base 32 also includes another passageway 32G which is coaxial with the aperture 32F and is of a similar internal diameter as the passageway 32F. The inner end of the passageway 32G merges with and is in fluid communication with the inner end of the passageway 32F. The passageway 32G terminates at its outer end in a tubular section which forms the port 22C. The tubular section forming the port 22C thus extends perpendicularly to the bottom surface of the cavity 32A and is centered on a longitudinally extending central axis A (FIG. 12). The body or base 32 includes another passageway 32H that extends parallel to the passageway 32F and is of a similar internal diameter as the passageway 32F. The inner end of the passageway 32H merges with and is in communication with a small passageway 32I (FIG. 13) that is in fluid communication with the cavity 32A. The passageway 32H terminates at its outer end in a tubular section which forms the port 22A. The top surface of the annular wall 32B includes an annular recess or groove 32J.

The cap 34 is a ring-like member formed of any suitable rigid material, e.g., ABS. It has an annular bottom section 34A which includes a helical internal thread 34B and an annular top section 34C which is of smaller external diameter than the bottom section and which includes a helical external thread 34D. The cap 34 is arranged to be screwed onto the body or base 32 to assemble the housing 28.

The diaphragm assembly 36 is best seen in FIGS. 4, 8, 12 and 13 and basically comprises a circular disk 36A and a center stiffener 36B. The disk 36A is formed of any suitable flexible material, e.g., silicone, and is a generally thin planar member having an annular ridge 36C projecting downward from its undersurface slightly radially inward of the periphery of the disk. The annular ridge 36C is shaped to fit closely within the annular recess to form a fluid-tight seal therewith. The central portion 36D of the disk is thickened and includes a central cavity in which the stiffener 36B is located. The stiffener is a stiff, disk-shaped member which serves as an anchor for the string 38. The string forms a portion of the valve assembly 46. The central portion 36D includes an aperture 36E through which the string 38 extends.

The internal threads 34B of the cap 34 are arranged to engage and screw onto the external threads 32C of the body or base 32 with the portion of the diaphragm disk 36A adjacent the periphery thereof tightly interposed between the cap and the body or base. In particular the annular ridge 36C of the disk 36A is located within the annular recess or groove 32J. Thus, when the cap is screwed onto the base the diaphragm disk closes off the chamber or cavity 32A, with the annular ridge tightly seated in the annular groove to form a good fluid-tight seal therebetween. The chamber or cavity 32A being coupled to the passageway 32I will thus be at the same positive pressure as existing within the laparoscopic field 10. Hence the chamber or cavity 32A will continuously monitor the pressure within the laparoscopic field 10.

The dial 30 is a circular disk-like member formed of any suitable rigid material, e.g., ABS. The dial is arranged to be rotated about the central longitudinal axis A of the housing 28 to establish a set point for the regulator device 22, i.e., the positive pressure level within the insufflated abdomen at which smoke will be evacuated. The dial has a downwardly extending peripheral sidewall 30A, the outer surface of which is ridged at 30B to provide a good gripping surface to enable the dial to be readily rotated about the axis A to the desired setting. The inner surface of the sidewall 30A includes a helical internal thread 30C which is configured to be screwed onto the external threads 34D of the cap. Thus, the dial can be rotated either clockwise or counterclockwise about the axis A to bring the undersurface of the cap either close to or further from the diaphragm assembly 36. With the dial screwed onto the housing, a cavity 30D is formed between the undersurface of the dial 30, the upper surface of the diaphragm 36A and the annular wall forming the top portion 34C of the cap 34. At least one aperture 30E is provided in the dial 30 in communication with the cavity 30D so that the pressure within the cavity is at atmospheric pressure. As best seen in FIG. 13, in the exemplary embodiment of the device 22 there are a large number of such apertures 30E in the cap 30. Those apertures are disposed about the dial close to its circular periphery.

The diaphragm assembly 36 is configured to be biased to establish the heretofore mentioned set point for the device 22. In particular, a helical compression spring 40 is located within the cavity 30D interposed between a spring base 42 and the top surface of the diaphragm disk 36A. The spring base 42 is a disk-like member having a central hub portion 42A configured to fit closely within the top end of the spring 40. A set screw 44 extends through a bore 30F in the dial 30 centered on the axis A. As best seen in FIG. 12 the bottom end of the set screw 44 is fixedly secured within a bore 42C in the spring base. The top end of the set screw includes an “Allen” wrench socket 44A for receipt of an Allen wrench (not shown) to adjust the set screw, i.e., moves it along axis A either closer to or further from the diaphragm assembly 36. Accordingly, rotation of the set screw about the axis A in the clockwise direction will cause the set screw to move closer to the diaphragm disk thereby trapping the spring between the spring base and the top surface of the diaphragm and compressing the spring somewhat. The set screw 44 serves to tune or set the initial load on the spring 40 to a factory designated setting during manufacturing so that the device will operate properly in the field.

As should be appreciated by those skilled in the art the presence of the disk base 42 decouples the rotation of the dial 30 from the spring 40. Thus, the rotation of the dial about the axis A does not result in the spring rotating with respect to the dial or to the diaphragm 36A, but does enable the spring to be compressed or decompressed (as the case may be) between the spring base and the diaphragm to whatever setting is desired to establish the set point for the regulator device 22. By decoupling of the spring from the dial one is able to ensure that the desired set point can be established and maintained accurately. In particular, the existence of the spring base 42 ensures that the spring will not be rotated with respect to the dial, upon rotation of the dial, since rotation of the spring if allowed could either coil the spring more tightly or uncoil the spring, depending upon the direction of rotation of the dial about the axis A. In either case rotation of the spring with respect to the dial will interfere with the normal operation of the spring. Moreover, the spring base 42 ensures that the spring will not rotate with respect to the diaphragm when the dial is rotated. This feature is also important, since rotation of the spring with respect to the diaphragm could apply a twisting action on the diaphragm, thereby interfering with its proper operation.

The valve assembly 46 basically comprises a movable valve member 46A and a stationary valve seat 46B. The movable valve member is in the form of piston having plural longitudinally extending ribs 46C terminating at a cone shaped lower end 46D. The string 38 is a very thin member formed of a flexible and relatively un-stretchable material, e.g. polyethylene. The upper end of the string is fixedly secured to the center stiffener 36B of the diaphragm assembly, with a mid-portion of the string passing through the aperture 32E, and with the bottom end of the string fixedly secured to the piston 46A. The valve seat 46B is a short section of a tube of any suitable resilient material, e.g., silicone, and includes a central passageway 46E. The valve seat 46B is fixedly secured within the passageway 32G at the bottom end thereof such that the bottom end of central passageway is at the port 22C. The conical lower end of the piston 46A is configured to be moved into engagement with the upper end of the valve seat 46B to close the valve and to be moved out of engagement with the upper end of the valve seat to open the valve. The movement of the valve member (piston) is accomplished by means of the string 38 which is connected to the diaphragm assembly. Thus, movement of the diaphragm disk 36A upward against the bias of the spring 40 will draw the string and the piston attached to it upward and out of engagement with the valve seat. Movement of the diaphragm downward will result in the movement of the piston downward and into engagement with the valve seat.

The amount of bias force provided by the spring 40 establishes the set point pressure at which the valve 46 opens. Thus, if the dial 30 is rotated to a position wherein the spring provides a bias force in excess of the force applied to the underside of the diaphragm 36A by the existing gas pressure within the cavity 32A (which is the pressure of the gas in the insufflated laparoscopic space), suction will not be applied to the laparoscopic field. However, once the dial is rotated to a position wherein the bias force applied by the spring is less than the force on the underside of the diaphragm, the diaphragm will flex upward against the bias of the spring thereby carrying the string and the valve member 46A upward off of the valve seat 46B, thereby opening the valve.

Use of the system 20 to evacuate smoke from the laparoscopic field is accomplished as follows. The dial 30 of the regulator device will be rotated to a desired position to establish a set point pressure at which the valve will open. That set point should be set to a pressure that is lower, e.g., 13 mm Hg, than the pressure of the insufflation gas, e.g., 15 mm Hg, supplied to the laparoscopic field by the insufflator. Thus, when the monitored pressure exceeds the set point (which will normally be the case since the set point is chosen to be less than the pressure of the insufflation gas) the valve 46A will automatically open to bring the passageway 32F and its associated port 22B into fluid communication with the passageway 32G and its associated port 22C. Accordingly, the suction applied at port 22C will draw smoke 4 from within the laparoscopic field through the trocar 18, the associated flexible tube 18A, and the port 22B to the vacuum source, thereby clearing the laparoscopic field of smoke.

The regulator device 22 operates continuously and automatically and limits the amount of pressure in the insufflated laparoscopic space to the set point pressure established by the rotational position of the dial 30. Thus, in the example above, if the insufflation pressure set by the insufflator is 15 mmHg and the set point of the device 22 is set to 13 mmHg, the amount of pressure existing within the laparoscopic space will be limited to 13 mmHg. This 13 mmHg pressure will be detected by the insufflator's pressure monitor (not shown) so that the insufflator will automatically attempt to raise the pressure within the laparoscopic field to the 15 mmHg to which the insufflator is set by pumping more gas at a faster rate into the laparoscopic field until the insufflator will be providing the maximum gas at the maximum rate. This action will continue as long as the insufflator is operating at its set point and the regulator device is operating at a lower set point, thereby resulting in the maximum rate of insufflation gas being introduced into the laparoscopic field and the concomitant maximum rate of evacuation of smoke from the laparoscopic field by the hospital's vacuum source.

It should also be noted that if the insufflator cannot keep up with the regulator device 22 to provide gas at the pressure set by the regulator device 22, e.g., 13 mmHg in the above example, the regulator device 22 will automatically stop. In particular, in such a case the pressure monitored by the port 22A will drop, whereupon the bias provided by the spring will overcome the bias provided by the pressure in the chamber 32A. This will cause the valve to close until the pressure within the chamber 32A again reaches the set point, e.g., 13 mmHg as the result of the insufflator pumping gas into the laparoscopic space. When that occurs, the valve will reopen to remove more smoke from the laparoscopic space. While such repeated opening and closing action will necessarily reduce the amount of smoke evacuated to the hospital's vacuum source, it will nevertheless prevent the laparoscopic field from being collapsed by the vacuum from that vacuum source. Thus, the regulator device 22 of the subject invention will enable whatever insufflator is used, be it a low flow rate insufflator or a high flow rate insufflator, to operate at its maximum capacity to insufflate the laparoscopic space with fresh gas while enabling smoke to be evacuated therefrom at the maximum rate that the insufflator is capable of achieving, thereby resulting in a visually clear laparoscopic space.

In the event that one of the trocars is removed from the laparoscopic field during operation of the system 20, or if there is a leak around one of the trocars extending into the laparoscopic field and the insufflator is not able to cope with the gas escaping through the aperture in the laparoscopic field at which leak is occurring or through which the trocar had extended, the system 20 will automatically shut down so that the hospital's vacuum source will not be applied to the laparoscopic field, thereby not exacerbating the collapse of the laparoscopic field.

When operation of the smoke evacuation system 20 is desired to be terminated, it can be accomplished easily. All that is required is to close the luer valve of the trocar 16 associated with the luer 16B by rotating the lever 16D or to close the luer valve of the trocar 18 associated with the luer 18B by rotating the lever 18D.

As mentioned above the cavity 32A includes four stand-offs 32D. These stand-offs serve to keep the area of the diaphragm against which the pressure of the gas entering into the cavity 32A constant irrespective the position of the diaphragm within that cavity. In particular, the stand-offs prevent the undersurface of the diaphragm portion 36D from resting directly on the bottom surface of the cavity 32. As should be appreciated by those skilled in the art, if the bottom surface of the diaphragm portion 36D was in direct contact with the bottom surface of the cavity that contact would decrease the available surface area of the diaphragm that the gas entering the cavity could act upon until the diaphragm lifts off of that surface, thereby interfering with the proper operation of the diaphragm assembly when the insufflation gas enters into that cavity via passageway 32I.

Turning now to FIG. 1B, there is shown alternative system 120 constructed in accordance with another aspect of this invention. The system 120 is particularly adapted for use in laparoscopic procedures in a confined laparoscopic space, e.g., within the rectum via the anus, by means of a gel port 100. The portions of the system 120 which are the same as those portions of the system 20 will be given the same reference numbers and the details of their construction and operation will not be reiterated in the interest of brevity.

The gel port 100 is a conventional device used for laparoscopic procedures in confined spaces, like the rectum. One suitable gel port for that purpose is that sold by Applied Medical Resources Corporation under the trademark GelPort Laparoscopic System. The gel port basically comprises a body configured for introduction into an orifice of the patient, e.g., the patient's anus. The gel port has two luer ports 102A and 102B, each of which is in fluid communication with the laparoscopic field within the patient's body, e.g., the rectum. The gel port 100 also includes a penetrable or piercable wall 104 formed of gel through which small trocars or instruments can be inserted.

The systems 20 and 120 of this invention (and in fact any system constructed in accordance with this invention) require at least three ports to the laparoscopic field, one port through which the insufflation gas is introduced into the laparoscopic field, one port through which the pressure of the insufflation gas within the laparoscopic field is monitored, and one port through which the smoke is extracted. Since the gel port 100 only includes two ports 102A and 102B, the system 120 also includes a smoke probe 50 to serve as one of the three ports. In particular, in the exemplary embodiment of the system 120, the port 102A serves as the insufflation port and is thus connected to the insufflator via the flexible tube 12A. The port 102B being in fluid communication with the laparoscopic field serves as the pressuring monitoring port and thus is connected to the device port 22A via the flexible tube 16A. The smoke probe 50 serves as the smoke evacuation port and is inserted through the gel wall 104. The exemplary system 120 also includes the trocar 14 through which the instrument 2 extends. The trocar 14 of the system 120 will typically be a shorter length trocar than that used for laparoscopic procedures in a less confined laparoscopic field, e.g., the abdomen of a patient and will be inserted through the get wall 104.

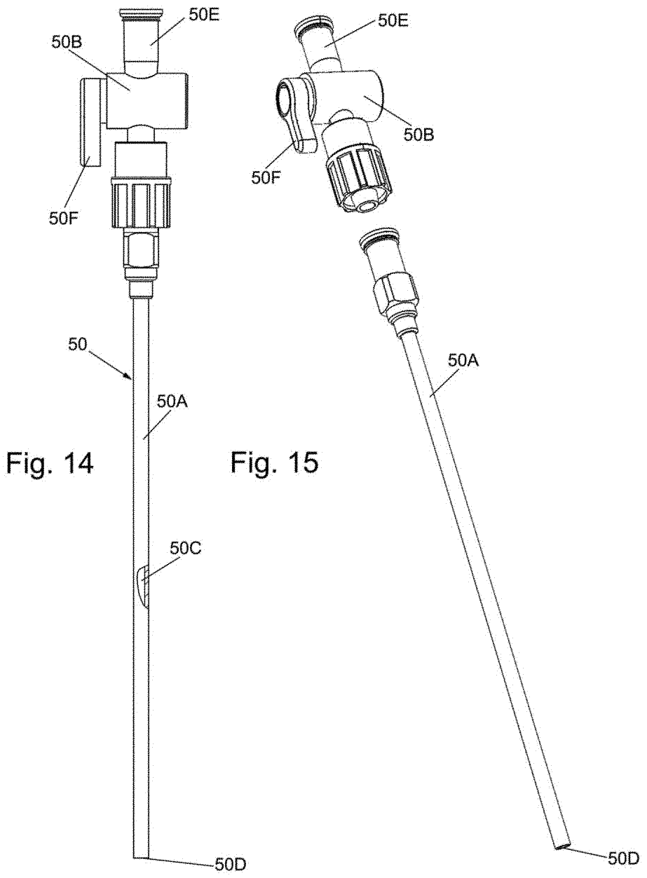

As best seen in FIGS. 14 and 15, the smoke probe 50 basically comprises an elongated tubular needle 50A, and a one-way luer stop cock 50B. The one-way luer stop cock 50B is configured to be connected to the device port 22B via the flexible tube 18A. The elongated tubular needle 50A includes a central passageway 50C extending therethrough and terminating in an open free end 50D of the needle. The needle 50A is configured to be inserted through (pierce) the piercable gel wall 104 of the gel port so that the open free end is located closely adjacent the source of the smoke 4. The one-way luer stop cock 50B includes a female luer connector 50E. A valve (not shown) is located within the one-way luer stop cock 50B between the proximal end of the passageway 50C and the female luer connector 50E. The valve is coupled to a pivotable lever 50F so that when the lever is pivoted to one rotational position the valve will be open and the female luer connector 50E will be in fluid communication with the passageway 50C. When the lever is pivoted to another rotational position the valve is closed to isolate the female luer connector from the passageway. The valve will be in the open position when the smoke probe is connected in the system 120 as shown in FIG. 1B and used to evacuate smoke from the laparoscopic field. During such use the system 120 will operate in the same manner as described with respect to system 20, except that the smoke will be evacuated from the laparoscopic field via the smoke probe 50 instead of via the trocar 18.

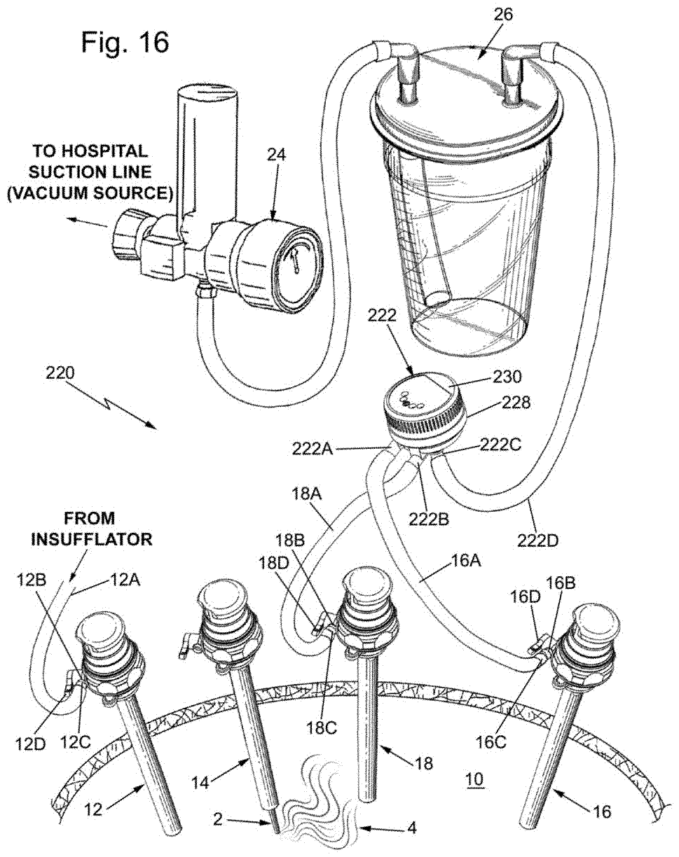

Referring now to FIG. 16 there is shown another exemplary system 220 for evacuating smoke from a laparoscopic field within the body of a patient constructed in accordance with this invention. The system 220 is similar to the system 20 shown in FIG. 1, but includes an alternative smoke evacuator regulator device 222 constructed in accordance with another aspect of this invention. In the interest of brevity the components of the system 220 that are the same as the components of the system 20 will be given the same reference numbers and the details of their construction, arrangement and operation will not be reiterated.

The smoke evacuator regulator device 222, like the smoke evacuator regulator device 22, is pneumatically-operated and configured to continuously monitor pressure of the insufflation gas within the laparoscopic field 10 and to automatically apply suction from the vacuum source e.g., a hospital's suction line, to the laparoscopic field when the pressure monitored reaches a preset level or set-point, whereupon smoke 4 within the laparoscopic field is evacuated from the laparoscopic field. Thus, the device 222 can be thought of as a regulator providing a controlled leak of gas and smoke from the laparoscopic field when the device is operating at its set-point. The set-point is adjustable within a range of values. Moreover, as will be described later the range itself is adjustable. That feature facilitates calibration of the device after it has been assembled and during testing.

The construction of the device 222 will be described in detail shortly. Suffice it for now to state that it includes three device ports, namely, a first device port 222A, a second device port 222B, and a third device port 222C. The first device port 222A is coupled to another trocar 16 via a flexible tube 16A. The trocar 16 is also a conventional device that includes a female luer connector 16B. The luer connector 16B is connected to one end of the flexible tube 16A by use of conventional male barbed connector 16C. An on-off valve (not shown) is located in the trocar 16 and is coupled to a pivotable lever 16D to either open or close the luer connector 16B, depending upon the rotational position of the lever. The trocar 16 extends into the laparoscopic field, e.g., the insufflated abdomen, to monitor the pressure of the gas therein. The second device port 222B is coupled to still another trocar 18 via a flexible tube 18A. The trocar 18 is also a conventional device that includes a female luer connector 18B. The luer connector 18B is connected to one end of the flexible tube 18A by use of conventional male barbed connector 18C. An on-off valve (not shown) is located in the trocar 18 and is coupled to a pivotable lever 18D to either open or close the luer connector 18B, depending upon the rotational position of the lever. The trocar 18 extends into the insufflated abdomen to serve as the means for evacuating the smoke 4 from the laparoscopic field 10. The third device port 222C is coupled to the vacuum source, e.g., the hospital's suction line, via a flexible tube 22D. In the exemplary system 220 shown in FIG. 1A a conventional suction regulator 24 and a conventional canister 26 are connected in series between the tube 222D and the vacuum source. The suction regulator and the canister are optional, and one or both need not be used, e.g., the tube 222D from the smoke evacuator regulator 222 can be directly connected to the vacuum source, if desired.

As will be described in detail later the smoke evacuator regulator device 222, when connected in the system 220 like shown in FIG. 16, operates to continuously monitor pressure of the gas within the laparoscopic field 10 via the trocar 16 and automatically applies suction from the vacuum source to the laparoscopic field via the trocar 18 when the pressure monitored reaches a preset level or set-point, e.g., 12 mm Hg. That action results in any smoke 4 within the laparoscopic field being evacuated from the laparoscopic field through the trocar 18, through its communicating tube 18A, and the device 222 to the vacuum source. Moreover, the regulator device 222 automatically maintains that level of pressure within the laparoscopic field to act as a controlled leak to continuously evacuate any smoke produced in that laparoscopic space out of the patient's body. The set-point is adjustable within an operating range of the device. Moreover, the operating range is itself adjustable from between a low value range (e.g., from 2 mm Hg to 22 mm Hg) to a high value range (e.g., from 10 mm Hg to 30 mm Hg) so that the device can be set to a desired operating range.

The smoke evacuator regulator device 222 basically comprises a housing assembly 228, a dial 230, a movable valve member 232, a diaphragm 234, a spring 236, an engagement member 238, a stationary valve seat 240, a spring base 242, and an adapter 244. The housing assembly 228 includes a body or base 246 and a cap 248. The body or base 246 is best seen in FIGS. 4, 9A and 9B and is a cup shaped member formed of any suitable rigid material, e.g., nylon or Acrylonitrile Butadiene Styrene (“ABS”). The body or base includes a recess 250 forming a pressure monitoring chamber or cavity bounded by an annular wall 252 extending about a central longitudinal axis X of the device 222. The annular wall includes a helical external thread 254 extending about the axis X. The thread 254 serves as the means for connecting the cap 248 onto the base 246, as will be described later.

The bottom wall of the base 246 includes an upstanding tubular projection 256 having a central passageway 258 extending therethrough and in fluid communication with the pressure monitoring chamber. The central passageway is in communication with a radially extending passageway 260 in the body or base 246. The passageway 260 terminates at its outer end in a tubular section 262 which forms the port 222B. The body or base 246 includes another tubular section 264 having a passageway 266 therein and which projects outward from the base parallel to the tubular section 262. The passageway 266 is of a similar internal diameter as the passageway 260 and is in fluid communication with the pressure monitoring chamber 250. The tubular section 264 forms the port 222A. A tubular collar 268 projects downward from the bottom of the base 246 centered about the axis X. The interior of the collar 268 is in fluid communication with the central passageway 258. The collar serves to mount the adapter 244 thereon via a bayonet type connection to be described later. The adapter 244 will also be described later. Suffice it for now to state that the adapter serves to mount the valve seat 240 at the bottom of the passageway 258 to enable a portion of the valve 232 (to be described later) to engage the valve seat when the valve is closed. The adapter includes a central passageway 270 extending through it to form the port 222C.

The cap 248 is a ring-like member formed of any suitable rigid material, e.g., nylon or ABS. It has an annular bottom section 274 and an annular top section 276. The bottom section 274 includes a helical internal thread 278 configured to be threadedly engaged by the helical external thread 254 of the base 246 to mount the cap on the base. The annular top section 276 is of smaller external diameter than the bottom section 274 and includes a helical external thread 280. The thread 280 is arranged to be engaged by a mating helical internal thread 282 (to be described later) forming a portion of the dial 230 to mount the dial on the cap and enable the dial to be rotated with respect to the cap to bring the dial closer or further away from the cap to establish the desired set-point. The threads 280 and 282 are oriented in the opposite direction from a normal left-handed oriented screw thread, such that rotation of the dial 230 in the clockwise direction will move the dial further away from the cap 248, whereas rotation of the dial in the counter-clockwise direction will move the dial closer to the cap.

The diaphragm 234 is best seen in FIGS. 19, 23A-23C, and 30A-30D. It basically comprises a circular disk formed of any suitable flexible material, e.g., silicone, having a central section 282, an intermediate section 284 surrounding the central section, and an outer section 286. The central section 282 is generally planar and of greater thickness that the intermediate and outer sections. The intermediate section 284 is of a generally U-shaped cross section. The outer section 286 is in the form of a generally planar flange projecting outward from the intermediate section. The diaphragm is mounted between the cap 248 and the base 246, with the flange of the diaphragm tightly interposed therebetween. In particular, as best seen in FIG. 15A the underside of the flange 286 immediately adjacent its peripheral edge includes an annular ridge 288 projecting downward. The annular ridge is shaped to fit closely within a correspondingly shaped annular recess 290 in the top surface of the annular wall 252 of the base 246. Thus, when the cap 248 is screwed onto the base 246 the diaphragm 234 closes off and forms a top wall of the chamber 250, with the annular ridge 288 tightly seated in the annular groove 290 to form a good fluid-tight seal therebetween. The chamber 250 being coupled to the passageway 266 will thus be at the same positive pressure as existing within the laparoscopic field 10. Hence the chamber 250 will continuously monitor the pressure within the laparoscopic field 10. The central section 282 of the diaphragm includes a central hole or opening 302 configured to receive a shaft section 304 (to be described later) of the movable valve member 232 and with the head section of the valve member disposed on top of the central section.

The dial 230 is a circular cup-shaped member formed of any suitable rigid material, e.g., nylon or ABS. The dial is arranged to be rotated about the central longitudinal axis X (also referred to as the rotation axis) of the housing 228 to establish a set-point for the device 222, i.e., the positive pressure level within the insufflated abdomen at which smoke will be evacuated. The top of the dial is planar and has a downwardly extending circular peripheral sidewall 292, the outer surface of which is ridged at 294 to provide a good gripping surface to enable the dial to be readily rotated about the axis X either clockwise or counterclockwise to the desired set-point. The inner surface of the sidewall 292 includes the heretofore identified helical internal thread 282, which as discussed earlier is configured to be screwed onto the external threads 280 of the cap 248. Thus, the dial can be rotated either clockwise or counterclockwise about the axis X to bring the undersurface of the cap either closer to or further from the diaphragm 234. As best seen in FIG. 15A when the dial 230 is screwed onto the cap 248 of the housing, a chamber 298 is formed between the undersurface of the dial, the upper surface of the diaphragm, the annular wall forming the top section 276 of the cap and the inner surface of the sidewall 294 of the cap. The chamber 298 forms a control pressure chamber of the smoke evacuator device 222 and is preferably at atmospheric pressure. Five apertures or holes 300A, 300B, 300C, 300D and 300E are located in the top wall of the dial and thus in fluid communication with the cavity 298 when the dial is mounted on the cap so that the pressure within the control pressure chamber 298 is at atmospheric pressure. The apertures are disposed in an arc extending about the central axis X and are equidistantly spaced from one another. As will be described later each of the apertures or openings 300A-300E also serves to receive the stop or set-screw 238 establish an operating range of set-points to which the smoke evacuator device 222 can be set.

The movable valve member 232 is arranged to be moved between a closed state and an open state and vice versa. In the closed state the port 222B is isolated from the port 222C. In the open state the port 222B is in communication with the port 222C, whereupon suction at the port 222C will clear smoke within the insufflation field out of that field through the device 222. The valve member is best seen in FIGS. 19, 22A-22C and 30A-30D and is formed of any suitable rigid material, e.g., nylon or ABS. It is of a generally thumb-tack like shape having the heretofore mentioned head 306, which is a generally planar disk-like member, from which the heretofore mentioned shaft 304 projects downward. The shaft is configured for sliding movement within the passageway 258 of the tubular projection 256 of the base member 246. To ensure a good fit of the shaft 304 within that passageway the shaft tapers downward from the head 306 at a very small angle, e.g., approximately 1 degree. The lower end 308 of the shaft 304 is planar and serves as the surface which will engage the valve seat 240 when the valve is in the closed state. A tapering bore 310 extends from the lower end 308 to a point adjacent the head of the valve member to ensure that the shaft will retain its desired size during the molding process used to make to valve member. An annular wall or ridge 312 extends upward from the top surface of the head 306 of the valve member and is centered about the axis X.

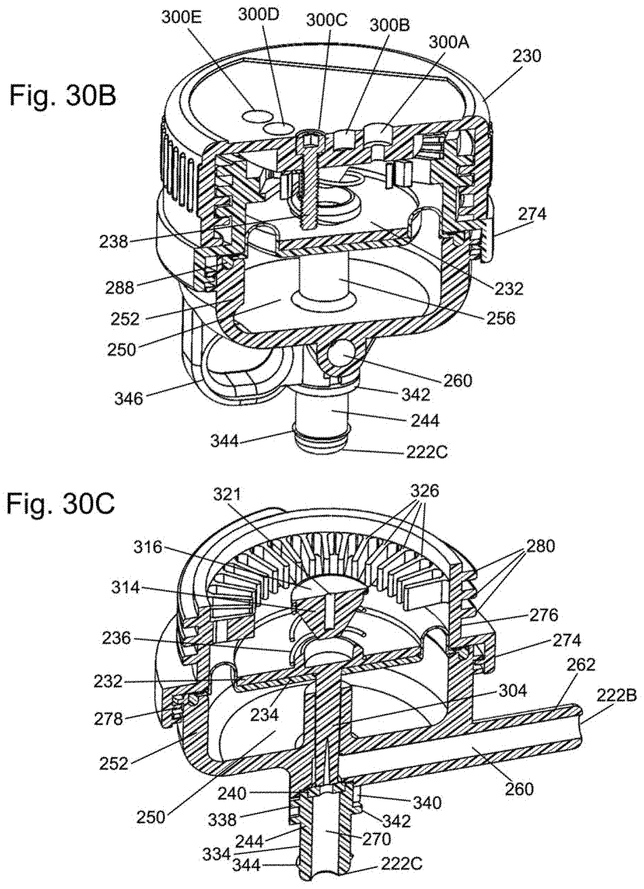

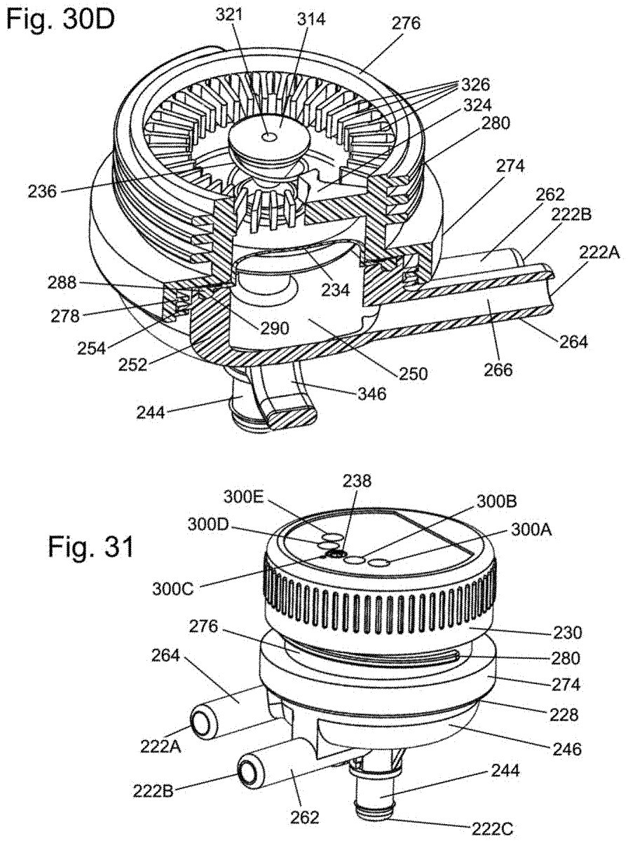

The diaphragm 234 is configured to be biased to establish the desired set-point for the device 222. To that end the spring 236, which is a helical compression spring, is located within the control pressure chamber 298 interposed between the spring base 242 and the head 306 of the valve member 232 as clearly shown in FIG. 30A. In particular the bottom of a conical portion 314 of the spring base 242 (to be described shortly) is located within the top end of the spring 236, with the annular wall or ridge 312 of the movable valve member 232 located within the bottom end of the spring. The head of the valve member 232 is disposed on top surface of the thickened central portion of the diaphragm 234, with the shaft 304 of the valve member extending through the hole 302 in the diaphragm and into the passageway 258 in the tubular projection 256 of the housing base 246.

The spring base 242 is best seen in FIGS. 19, 26A-26B, and FIGS. 30A-30D. In particular, the spring base is a plug-shaped member formed of any suitable rigid material, e.g., nylon or ABS. The bottom portion of the spring base forms the heretofore identified conically shaped surface 314. The top portion of the spring base is in the form of a planar top surface 316 from which a flange 318 projects outward. The undersurface of the flange merges with a circular outer surface to form a recess 320 at the interface with the conical surface 314. The outer diameter of the recess 320 is slightly larger than the inner diameter of the top of the helical spring so that the top of the helical spring can be located within the recess like shown in FIG. 30A. A central bore 321 extends into the spring base from the planar top surface. The central bore 321 is configured to receive a post 322 projecting downward from the inner surface of the top of the dial 230 coaxial with central axis X so that the spring base can freely rotate about the post.

As should be appreciated by those skilled in the art the presence of the spring base 242 decouples the rotation of the dial 230 from the spring 236. Thus, the rotation of the dial about the axis X does not result in the spring rotating with respect to the dial, but does enable the spring to be compressed or decompressed (as the case may be) between the spring base and the head 306 of the valve member 232 to whatever setting is desired to establish the set-point for the device 222. By decoupling of the spring 236 from the dial 230 one is able to ensure that the desired set-point can be established and maintained accurately. In particular, the existence of the spring base 242 ensures that the spring will not be torqued or rotated with respect to the dial, upon rotation of the dial, since rotation of the spring if allowed could either coil the spring more tightly or uncoil the spring, depending upon the direction of rotation of the dial about the axis X. In either case rotation of the spring with respect to the dial will interfere with the normal operation of the spring. Moreover, the spring base 242 ensures that the spring will not rotate with respect to the diaphragm when the dial is rotated. This feature is also important, since rotation of the spring with respect to the head of the valve and the underlying portion of the diaphragm, which action could apply a twisting action on the diaphragm, thereby interfering with its proper operation.

The movement of the valve member 232 is effected by the movement of the diaphragm 234 under the force applied to the underside of the diaphragm by the pressure within the chamber 250 and against the bias provided by the spring 236. Thus, movement of the diaphragm upward against the bias of the spring will draw the flat end surface 308 of the valve member off of the valve seat 240 when the pressure applied to the underside of the surface reaches the set-point. As discussed above, the amount of bias force provided by the spring establishes the set-point pressure at which the valve opens. Thus, if the dial is rotated to a position wherein the spring provides a bias force in excess of the force applied to the underside of the diaphragm by the existing gas pressure within the chamber 250 (which is the pressure of the gas in the insufflated laparoscopic space), the bottom surface 308 of the valve member will be in engagement with the valve seat thereby isolating port 222C from port 222B so that suction will not be applied to the laparoscopic field. However, once the dial is rotated to a position wherein the bias force applied by the spring is less than the force on the underside of the diaphragm, the diaphragm will flex or otherwise move upward against the bias of the spring thereby carrying the valve member upward off of the valve seat, thereby opening the valve.

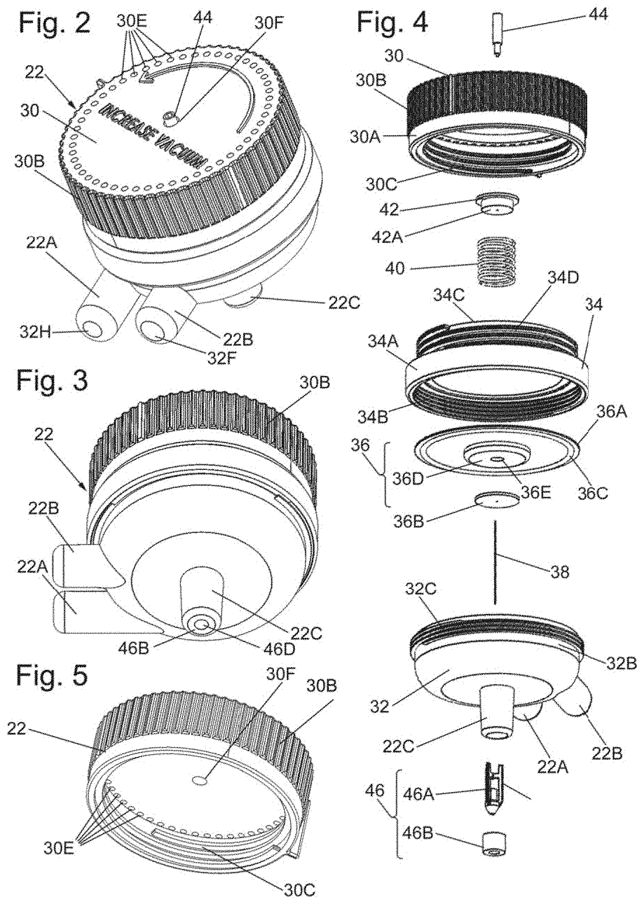

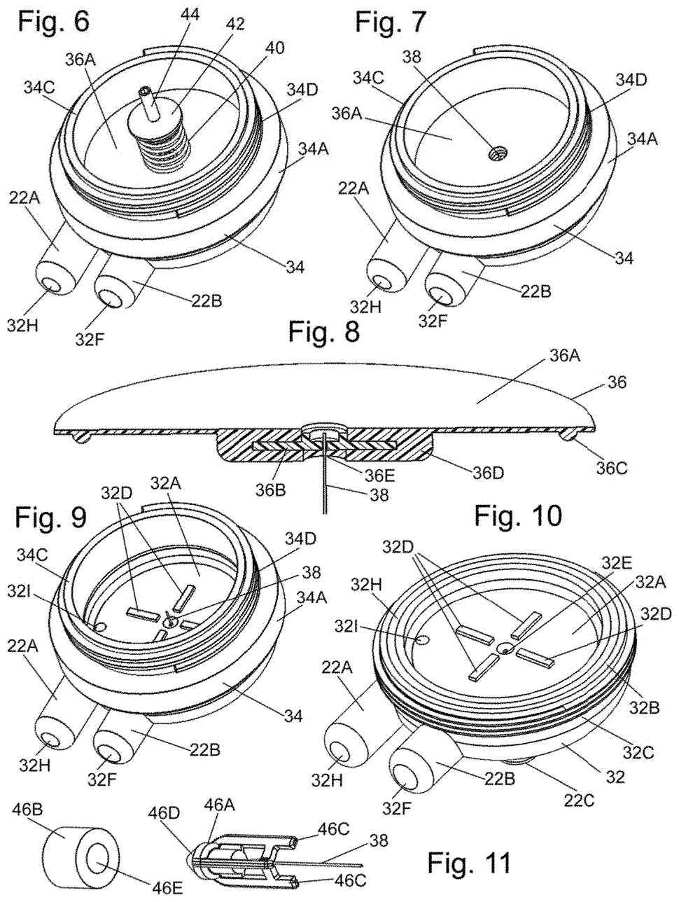

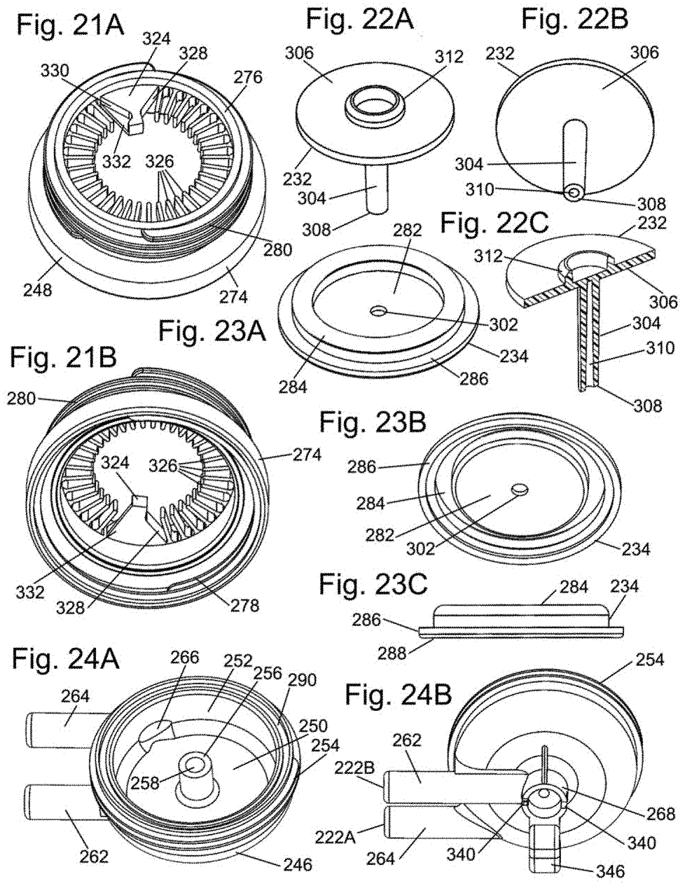

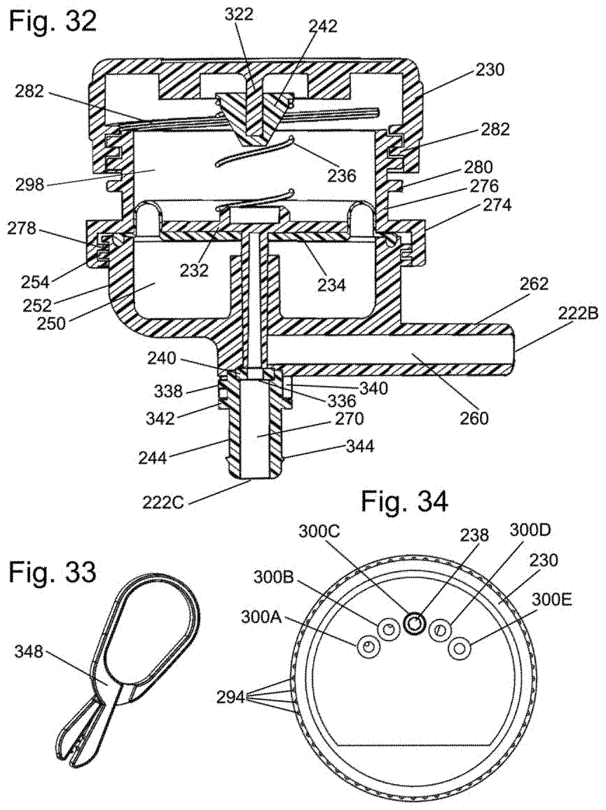

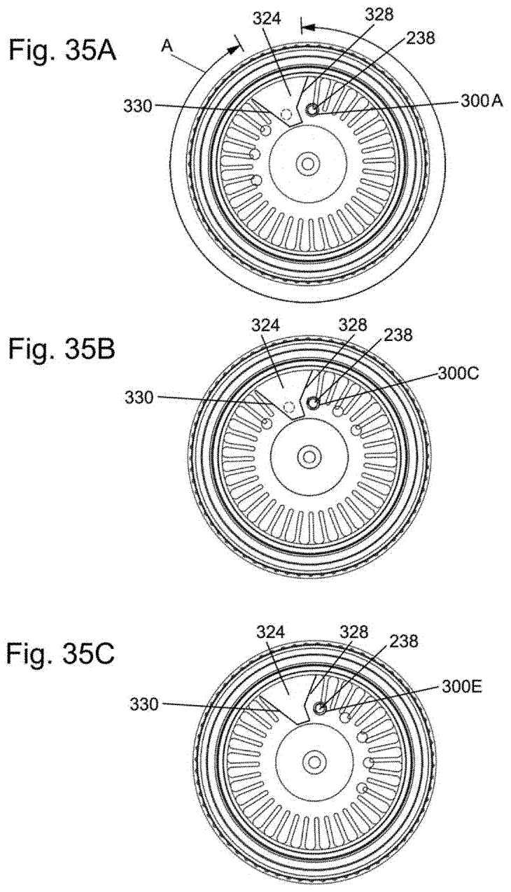

The dial 230 is rotatable through an arc A (FIG. 35A) of approximately 350 degrees about the axis X from a “start” or “down” position (like shown in FIGS. 2 and 3) to a “stop” or “up” position (like shown in FIGS. 31 and 32) to establish the operating range of set-points to which the smoke evacuation device 222 can be set. Moreover, the operating range itself can be shifted up or down to enable the operating range to be precisely set at the factory when the smoke evacuator device is assembled and tested. In particular, the start and stop positions are established by a stop member 324 forming a portion of the cap 248 in cooperation with the engagement member 238. The engagement member 238 is an elongated member which is configured to be located within one of the holes 300A-300E to engage the stop member 324. In the exemplary embodiment shown the engagement member is a set-screw formed of any suitable material, e.g., stainless steel. The top end or head of the set-screw 238 includes an “Allen” wrench socket for receipt of an Allen wrench (not shown) to secure the set-screw in any of the holes 300A-300E. While not shown, each of the holes into which the set-screw is to be located is tapped. The stop member is best seen in FIGS. 21A and 21B and is in the form of a projection located between a series a radially extending fins 326, whose construction and operation will be described later. The stop member 324 includes first side 328, which will be referred to as the “start” side and a second and oppositely located side 330, which will be referred to as the “stop” side. The start side 328 is configured to be engaged by the set-screw 238 when the dial 230 is rotated in the counterclockwise direction to bring the dial to its closest position with respect to the cap 248, i.e., when the dial is in the “start” position. Conversely, the stop side 330 is configured to be engaged by the set-screw 238 when the dial is rotated in the clockwise direction to bring the dial to its furthest position with respect to the cap, i.e., when the dial is in the “stop” position. Thus, the rotation of the dial in the clockwise direction from the start position towards the stop position will establish the particular set-point within the range established by the start and stop points. Moreover, the engagement of the set-screw with the stop side of the stop member will preclude the dial from becoming screwed off of the cap. The stop member 324 is reinforced by a bracing wall 332 contiguous with the stop side 330, so that engagement of the stop member by the set-screw will not result in breaking the stop member from the cap.

As mentioned earlier the cap includes a series of fins 326. Each fin is a thin blade-like member projecting inward radially from the inner surface of the top section 276. The fins are equidistantly spaced from one another, with the free end of each fin being located slightly beyond (inward of) the arc along which the adjustment holes 300A-300E are disposed. Accordingly, rotation of the dial about the axis X will bring the free end of the set-screw into engagement with the free ends of the fins as the dial is rotated in either rotational direction, whereupon the fin will flex and then snap back to its original shape thereby creating a clicking sound. The cooperation of the engagement member (set-screw 238) and the fins forms a detent mechanism. The detent mechanism ensures that when the rotation of the dial is stopped at any rotational position to establish the desired set-point, the dial will be retained in that rotational position by the engagement of the set-screw with the particular fin at that rotational position. As mentioned earlier, and in accordance with one preferred aspect of this invention, the operating range of the smoke evacuator regulator device 222 is adjustable up or down as a result of the position of the set-screw in any of the adjustment holes 300A-300E. In particular, the starting height position of the dial with respect to the cap 248 and the ending height position of the dial with respect to the cap, and hence the amount of bias provided by the spring 236, is established by the position of the set-screw 238 in any one of those adjustment holes. The particular hole that the set-screw is located in establishes the operating range for the device, i.e., the lowest set-point and the highest set-point. Irrespective of which adjustment hole the set-screw is located in, when the dial is in the start or down position the set-screw 238 will abut the start side 328 of the stop member 324 like shown in FIGS. 35A-35C and the dial 230 will be located closest to the cap 248, whereupon the spring 236 will be compressed to establish the highest set-point for that particular operating range. In the stop or up position, the set-screw abuts the stop side 330 of the stop member so that the dial is located furthest from the cap, whereupon the spring will establish the lowest set-point for that particular operating range.

The exemplary embodiment of the smoke evacuation regulator device 222 has an operating range of approximately 20 mmHg. Thus, the rotation of the dial in the clockwise direction from its “start” position to its “stop” position will reduce the set-point approximately 20 mmHg. Conversely, the rotation of the dial in the counterclockwise direction from its “stop” position to its “start” position will increase the set-point approximately 20 mmHg. The spacing of the adjustment holes 300A-300E with respect to one another establishes a difference of approximately 2 mmHg when the set-screw 238 is moved from one adjustment hole to an immediately adjacent adjustment hole. Thus, for example the positioning the set-screw in the hole 300A, like shown in FIG. 35A will establish an operating range of approximately 2 to 22 mmHg, whereas the positioning the set-screw in the hole 300E, like shown in FIG. 35C, will establish an operating range of approximately 10 to 30 mmHg, and the positioning of the set-screw in the hole 18C, like shown in FIGS. 34 and 35B, will establish an operating range of approximately 6-26 mmHg.

In practice, the smoke evacuator regulator device 222 will typically be set up to have an operating range of approximately 6 to 26 mmHg. To that end, when the device is assembled the set-screw 238 is located in the center adjustment hole 300C and then the device is tested and calibrated to make sure that it operates at that range and to adjust it up or down (calibrate it, if necessary) to operate in that range. In particular, with the set-screw in the center adjustment hole 300C the suction port 222C will be connected via tube 222D to a source of suction simulating the suction arrangement shown in FIG. 16. The pressure sensing port 222A will be connected via tube 16A to a tank simulating a patient's insufflated abdomen which is insufflated to a pressure of approximately 15 mmHg by an insufflator. The smoke evacuation port 222B will be connected via tube 18A to the tank simulating a patient's insufflated abdomen. The dial 230 of the device 222 will then be rotated clockwise slowly from the stop position and the pressure within the tank measured. When the pressure within the tank reaches 13 mmHg, the device's valve will open to provide a controlled leak to vent the gas from the tank through the device to the suction source. At that point the rate of flow of vented gas can be measured and recorded to meet a target of 20 liters/minute. After that has been accomplished the pressure of gas provided by the insufflator will be increased to 30 mmHg, whereupon and the dial 230 of the device 222 will be rotated in the clockwise direction until it reaches its stop or up position to take a “low” reading. That reading should preferably be 6 mmHg and no less than 2 mmHg. If, for example, the reading is 8 mmHg, which might occur due to manufacturing or assembly intolerances of the components of the device 222, the device will be recalibrated to the desired operating range. That is achieved by moving the set-screw one hole to the left (counterclockwise), i.e., to hole 300B, which allows the dial when rotated clockwise to be moved to an up position further away from the cap before it reaches the stop position, thereby decreasing the bias force provided by the spring, to produce a controlled leak shifting the operating range down by 2 mmHg. Thus, the low set-point will be the desired 6 mmHg and the new (calibrated) operating range will be the desired 6-26 mmHg.

In short, the positioning of the set-screw from one adjustment hole to another of the adjustment holes results in the repositioning of the dial with respect to the base and a corresponding change in the compression of the spring. Even though the dial will be rotated through the same total angular rotation, i.e., through arc A, from the start position to the stop position the distance of the dial with respect to the cap will be changed and hence the range of compression or bias provided by the spring will be shifted up or down depending upon the adjustment hole into which the set-screw is placed.

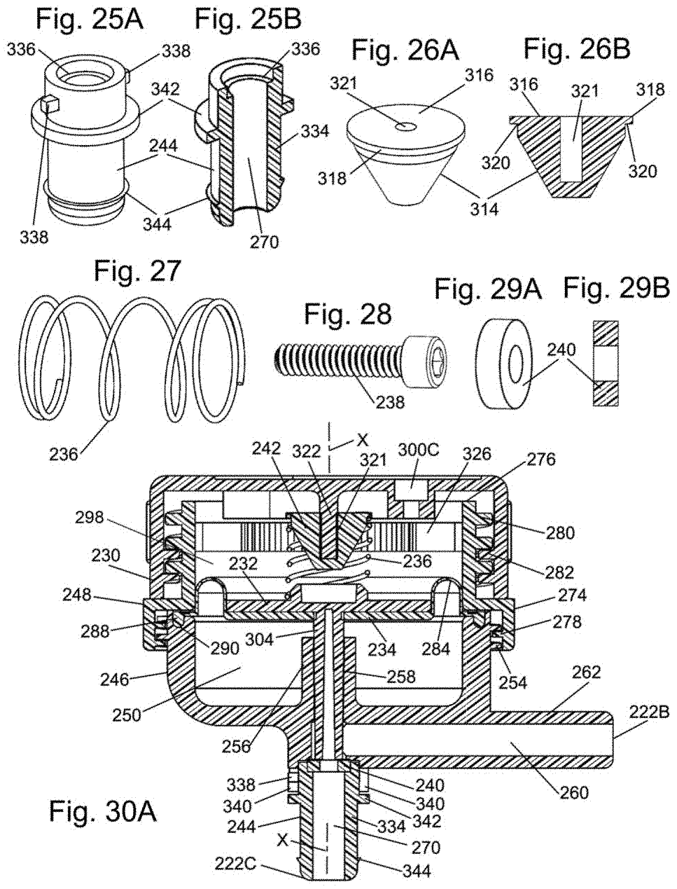

Turning now to FIGS. 19, 29A, 29B, 30A and 30C, the valve seat 240 will now be described. It basically consists of a square profile O-ring formed of any suitable elastomeric material, e.g., silicone. Being of square profile it includes a planar top surface and a planar bottom surface. As mentioned earlier the valve seat is mounted in the adapter 244, which in turn is secured to the collar 268 of the base 246. The adapter 244 is best seen in FIGS. 19, 25A, 25B, and 30A-30D, and is a tubular member formed of any suitable rigid material, e.g., nylon or ABS. As mentioned earlier the adapter includes a central passageway 270 forming the suction port 222C. The passageway 270 is bounded by a circular sidewall 334. The top end of the sidewall includes an annular recess 336, which is configured to receive the valve seat 240 therein. A pair of short height pegs 338 projects outward diametrically from the sidewall 334 adjacent the top end thereof. The pegs are configured to fit into respective ones of L-shaped slots 340 (FIGS. 30A and 30C) in the collar 268 of the base 246 to form a bayonet-like connection between the adapter 244 and the base. In particular, each L-shaped slot 340 includes a vertically oriented linear entry portion extending upward from the bottom surface of the collar, and a horizontally extending linear portion merging with the upper end of the entry portion. The bayonet-like connection serves to fixedly secure the adapter to the base after the valve seat has been located in the recess 336 of the adapter. To that end, each of the pegs 338 is aligned with a respective one of the entry portions of the L-shaped slots 340 and the adapter pushed upward to cause the pegs 338 to enter those entry portions. When the pegs reach the ends of the entry portions the adapter is rotated about the axis X to cause the pegs to enter the horizontally extending linear portions of the L-shaped slots. That action completes the connection of the adapter to the base and tightly interposes the valve seat between the adapter and the base. The adapter also includes an annular ridge 342 extending about the periphery of the sidewall 334 to act as a stop during the connection of the adapter to the collar of the base. An annular barb 344 extends about the periphery of the sidewall 334 close to the free end of the adapter so that when the free end of the adapter is inserted into an open end of the suction tubing 222D the tubing is secured thereto in a good fluid-tight seal.

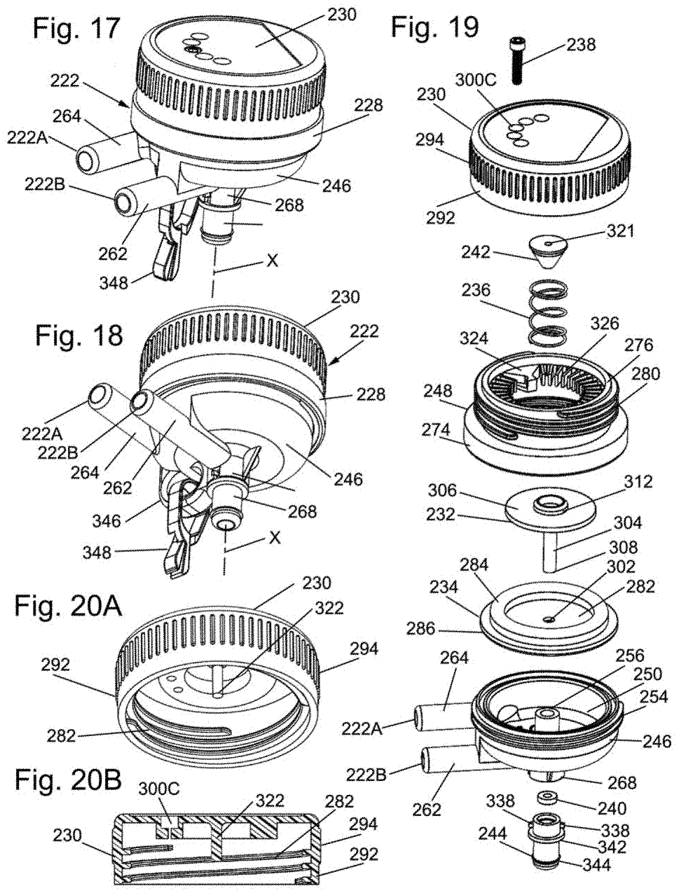

As can be seen in FIGS. 18 and 30B the base 246 of the housing includes a ring or loop 346. The ring or loop serves to mount a conventional sheet or drape clip 348 thereon. The drape clip 348 is shown in FIGS. 17, 18 and 33 and serves to enable the smoke regulator device 222 to be releasably secured to a sterile drape or sheet within the surgical field. That feature ensures that the device 222 is readily accessible to the surgeon or other personnel within the operating field if the device is to be accessed, e.g., adjusted, during the laparoscopic procedure.

Use of the system 220 to evacuate smoke from the laparoscopic field is accomplished as follows. With the system set up like shown in FIG. 16, and all of the trocar on/off valves open the dial 230 of the smoke evacuator regulator device 222 is rotated clockwise from its start position to a desired position to establish a set-point pressure at which the valve will open. That set-point should be set to a pressure that is lower, e.g., 12 mm Hg, than the pressure of the insufflation gas, e.g., 15 mm Hg, supplied to the laparoscopic field by the insufflator. Thus, when the monitored pressure exceeds the set-point (which will normally be the case since the set-point is chosen to be less than the pressure of the insufflation gas) the valve 232 will automatically open, i.e., its planar end 308 will move off of the valve seat 240 to bring the passageway 260 and its associated port 222B into fluid communication with the passageway 270 and its associated suction port 222C of the adapter 244. Accordingly, the suction applied at port 222C will draw smoke 4 from within the laparoscopic field through the trocar 18, the associated flexible tube 18A, and the port 222B to the vacuum source, thereby clearing the laparoscopic field of smoke.

The smoke evacuator regulator device 222 operates continuously and automatically to limit the amount of pressure in the insufflated laparoscopic space to the set-point pressure established by the rotational position of the dial 230. Thus, in the example above, if the insufflation pressure set by the insufflator is 15 mmHg and the set-point of the device 222 is set to 12 mmHg, the amount of pressure existing within the laparoscopic space will be limited to 12 mmHg. This 12 mmHg pressure will be detected by the insufflator's pressure monitor (not shown) so that the insufflator will automatically attempt to raise the pressure within the laparoscopic field to the 15 mmHg to which the insufflator is set by pumping more gas at a faster rate into the laparoscopic field until the insufflator will be providing the maximum gas at the maximum rate. This action will continue as long as the insufflator is operating at its set-point and the regulator device is operating at a lower set-point, thereby resulting in the maximum rate of insufflation gas being introduced into the laparoscopic field and the concomitant maximum rate of evacuation of smoke from the laparoscopic field by the hospital's vacuum source.

It should also be noted that if the insufflator cannot keep up with the smoke evacuation regulator device 222 to provide gas at the pressure set by the device, e.g., 12 mmHg in the above example, the device will automatically stop. In particular, in such a case the pressure monitored by the port 222A will drop, whereupon the bias provided by the spring will overcome the bias provided by the pressure in the chamber 250. This will cause the valve to close until the pressure within the chamber 250 again reaches the set-point as the result of the insufflator pumping gas into the laparoscopic space. When that occurs, the valve will reopen to remove more smoke from the laparoscopic space. While such repeated opening and closing action will necessarily reduce the amount of smoke evacuated to the hospital's vacuum source, it will nevertheless prevent the laparoscopic field from being collapsed by the vacuum from that vacuum source. Thus, the smoke evacuation device 222 of the subject invention acts as controlled leak or regulate to enable whatever insufflator is used, be it a low flow rate insufflator or a high flow rate insufflator, to operate at its maximum capacity to insufflate the laparoscopic space with fresh gas while enabling smoke to be evacuated therefrom at the maximum rate that the insufflator is capable of achieving, thereby resulting in a visually clear laparoscopic space.

In the event that one of the trocars is removed from the laparoscopic field during operation of the system 220, or if there is a leak around one of the trocars extending into the laparoscopic field and the insufflator is not able to cope with the gas escaping through the aperture in the laparoscopic field at which leak is located or through which the trocar had extended, the system 220 will automatically shut down so that the hospital's vacuum source will not be applied to the laparoscopic field, thereby not exacerbating the collapse of the laparoscopic field.

When operation of the smoke evacuation system 220 is desired to be terminated, it can be accomplished easily. All that is required is to close the luer valve of the trocar 16 associated with the luer 16B by rotating the lever 16D or to close the luer valve of the trocar 18 associated with the luer 18B by rotating the lever 18D.

While not shown the system systems of this invention making use of a smoke evacuator regulator device 22 or 222 or any other regulator device constructed in accordance with this invention may include a component that displays or shows that the smoke evacuator system is actively pulling gas (smoke) from the laparoscopic field. That component may be a flow indicator or pressure indicator. The flow indicator can be located in two different locations, namely, between the trocar for evacuating the smoke and the regulator device or between the hospital vacuum source and the regulator device. A pressure indicator can only be located between the trocar for evacuating the smoke and the regulator device. In fact, it is contemplated that either the flow indicator or the pressure indicator be part of the regulator device, e.g., be to included in the housing at the appropriate port.

It should be pointed out that a regulator device in accordance with this invention can be constructed differently than the exemplary embodiments 22 and 222 described above, providing that it includes a port for monitoring the pressure with the laparoscopic field, an evacuation port configured for coupling to a vacuum source, and a valve for automatically coupling the vacuum source to the evacuation port when the monitored pressure within the laparoscopic field reaches a preset (set-point) level.

Without further elaboration the foregoing will so fully illustrate our invention that others may, by applying current or future knowledge, adopt the same for use under various conditions of service.

We claim:

1. A smoke evacuator regulator device configured for coupling to a vacuum source for evacuating smoke from a laparoscopic field within the body of a patient, the laparoscopic field being insufflated with insufflation gas under positive pressure, said pneumatic-operated regulator device being pneumatically-operated and configured to continuously monitor pressure of the gas within the laparoscopic field and to automatically apply suction from the vacuum source to the laparoscopic field when the pressure monitored reaches a set-point, whereupon smoke within the laparoscopic field is evacuated from the laparoscopic field.

2. The smoke evacuator regulator device of claim 1, wherein said smoke evacuator regulator device comprises:
a housing;
a first device port in said housing and configured for coupling to the laparoscopic field for monitoring the pressure of the gas within the laparoscopic field;
a second device port in said housing and configured for coupling to the laparoscopic field for evacuating smoke from the laparoscopic field via said second port;
a third device port in said housing and configured for coupling to the vacuum source; and
a valve in said housing, said valve being in a normally closed state and coupled between said second device port and said third device port, said valve being operative in automatic response to the pressure of the gas monitored at said first device port, whereupon said valve opens to an open state to enable suction from the vacuum source to be applied through said third device port to said second device port when the pressure monitored at said first device port reaches said set-point, whereupon smoke within the laparoscopic field is evacuated from the laparoscopic field via said second device port and said third device port.

3. The smoke evacuator regulator device of claim 2, additionally comprising;
a pressure chamber in fluid communication with said first port; and
a movable diaphragm forming a portion of said pressure chamber and coupled to said valve, said movable diaphragm being biased to apply a bias force in opposition to pressure within said pressure chamber, said bias force establishing said set-point.

4. The smoke evacuator regulator device of claim 3, wherein said bias force is adjustable.

5. The smoke evacuator regulator device of claim 4, wherein said bias force is established by a spring.

6. The smoke evacuator regulator device of claim 1, wherein said smoke evacuator regulator device comprises:
a housing including a pressure monitoring chamber, a first port, a second port, a third port, said pressure monitoring chamber being configured for fluid communication with the laparoscopic field via said first port, whereupon some of the insufflation gas is within said pressure monitoring chamber, said second port being configured for coupling to the laparoscopic field for evacuating smoke from the laparoscopic field via said second port, said third port being configured for coupling to the vacuum source;
a diaphragm establishing a wall of said pressure monitoring chamber and being movable in response to a force applied thereto by the pressure of the insufflation gas within said pressure monitoring chamber;
a rotatable dial coupled to said housing and rotatable through an arc about a rotation axis between a first angular position and a second angular position, and vice versa, to establish an operating range for said smoke evacuator device;
an engagement member coupled to said rotatable dial and configured to cooperate with a stop member to adjust said operating range up or down to a desired operating range:
a spring coupled to said rotatable dial and configured to apply a bias force to said diaphragm in opposition to the force applied by the pressure of the insufflation gas within said pressure monitoring chamber, said bias force being adjustable within said desired operating range in response to rotation of said dial about said axis between said first angular position and said second angular position to establish said set-point, said set-point being adjustable within said desired operating range; and
a valve comprising a movable valve member and a valve seat in said housing, said valve being normally in a closed state isolating said second port from said third port, said movable valve member being connected to said movable diaphragm and movable therewith in automatic response to the pressure of the insufflation gas in said pressure monitoring chamber, whereupon said valve opens to an open state to enable suction from the vacuum source to be applied through said third port to said second port when the gas pressure monitored by said pressure monitoring chamber reaches said set-point, whereupon smoke within the laparoscopic field is evacuated from the laparoscopic field via said second port and said third port.

7. The smoke evacuator regulator device of claim 6, wherein said stop member is located within said housing, wherein said rotatable dial includes plural spaced apart openings extending in an arc about said rotation axis, and wherein said engagement member is configured to be located in any of one said openings to establish said desired operating range.

8. The smoke evacuator regulator device of claim 7, wherein said stop member includes a first surface and a second surface, and wherein said engagement member is configured to engage said first surface at said first angular position and to engage said second surface at said second angular position.

9. The smoke evacuator regulator device of claim 6, additionally comprising a control pressure chamber within said housing configured to be at atmospheric pressure and defined between said rotatable dial and said diaphragm, said rotatable dial being configured to move toward said diaphragm by the rotation of said rotatable dial in a first rotational direction about said rotational axis and to move away from said diaphragm by the rotation of said dial in a second and opposite rotation direction, said spring being interposed between said rotatable dial and said diaphragm in said control pressure chamber, whereupon said bias force provided by said spring is increased upon rotation of said dial in said first rotational direction and said bias force provided by said spring is decreased upon rotation of said dial is said second and opposite rotational direction.

10. The smoke evacuator regulator device of claim 9, wherein said housing comprising a base and a cap, wherein said diaphragm is interposed between said base and said cap, wherein said spring is interposed between said rotatable dial and said diaphragm, wherein said rotatable dial is threadedly secured to said cap.

11. The smoke evacuator regulator device of claim 10, wherein said spring comprises a helical compression spring having a longitudinal axis, said spring being interposed between said rotatable dial and a portion of said diaphragm, with said longitudinal axis of said spring being coaxial with said rotation axis.

12. The smoke evacuator regulator device of claim 11, additionally comprising a rotatable isolation disk interposed between said rotatable dial and said spring, whereupon rotation of said rotatable dial in either said first or second direction about said rotation axis does not cause said spring to rotate about said rotation axis.

13. The smoke evacuator regulator device of claim 6, wherein said valve seat is formed of a resilient material, said movable valve member including an end surface configured to engage said valve seat when said valve is in said normally closed state, and to be disengaged from said valve seat when said valve is in said open state.

14. The smoke evacuator regulator device of claim 6, wherein said rotatable dial includes a detent mechanism for holding said rotatable dial at any rotational position establishing said set-point.

15. The smoke evacuator regulator device of claim 14, wherein said detent mechanism comprises plurality of radially extending fins configured to be engaged by said engagement member to hold said rotatable dial at any rotational position establishing said set-point.

16. The smoke evacuator regulator device of claim 14, wherein the smoke evacuator regulator device forms a portion of a system comprising:
a first tube configured to be connected to a first trocar extending into the laparoscopic field;
a second tube configured to be connected to a second trocar extending into the laparoscopic field; and
a third tube configured to be connected to a canister which is connected to a vacuum source.

17. A system for evacuating smoke from a laparoscopic field within the body of a patient, the laparoscopic field being insufflated with insufflation gas under positive pressure, said system comprising
a first instrument port in fluid communication with the laparoscopic field for monitoring the pressure of the gas in the laparoscopic field;
a second instrument port in fluid communication with the laparoscopic field for evacuating smoke from the laparoscopic field via said second instrument port;
a smoke evacuator regulator device comprising:
a housing;
a first device port in said housing and configured for coupling to said first instrument port for monitoring the pressure of the gas in the laparoscopic field;
a second device port in said housing and configured for coupling to said second instrument port for evacuating smoke from the laparoscopic field via the second device port;
a third device port in said housing and configured for coupling to a vacuum source; and
a valve in said housing, said valve being in a normally closed state and coupled between said second device port and said third device port, said valve being operative in automatic response to the pressure of the gas monitored at said first device port, whereupon said valve opens to an open state to enable suction from the vacuum source to be applied through said third device port to said second device port when the pressure monitored at said first device port reaches a set-point, whereupon smoke within the laparoscopic field is evacuated from the laparoscopic field via said second device port and said third device port.

18. The system of claim 17, wherein said smoke evacuator regulator device is pneumatically-operated and additionally comprises:
a pressure chamber in fluid communication with said first device port; and
a movable diaphragm forming a portion of said pressure chamber and coupled to said valve, said movable diaphragm being biased to apply a bias force in opposition to pressure within said pressure chamber, said bias force establishing said set-point.

19. The system of claim 18, wherein said bias force is adjustable.

20. The system of claim 19, wherein said bias force is established by a spring.

21. The system of claim 17, wherein said first instrument port forms a portion of a first instrument extending into the laparoscopic field, and wherein said second instrument port forms a portion of a second instrument extending into the laparoscopic field.

22. The system of claim 21, wherein said first instrument is a trocar and wherein said second instrument is a trocar.

23. The system of claim 17, wherein the laparoscopic field is confined, and wherein said system comprises a gel port and a smoke evacuating probe, said gel port including said first instrument port, and another instrument port configured to be coupled to an insufflator supplying an insufflation gas under positive pressure to the laparoscopic field via said other instrument port, said gel port including a penetrable portion, said second instrument port comprising a portion of said smoke evacuating probe, said smoke evacuating probe comprising an elongated needle and a one-way luer stop cock, said elongated needle having a longitudinal passageway extending therethrough, said one-way luer stop cock including a luer connector and a movable lever, said luer connector being configured to be brought into fluid communication with said longitudinal passageway when said movable lever is moved to a predetermined position, said elongated needle being configured for penetrating said penetrable portion of said gel port.

24. A method of evacuating smoke from a laparoscopic field within the body of a patient, the laparoscopic field being insufflated with gas under positive pressure, said method comprising:
continuously monitoring pressure of the insufflation gas within the laparoscopic field via a first instrument port in fluid communication with the laparoscopic field;
coupling a pneumatically-operated smoke evacuator regulator device to a vacuum source and to a second instrument port in fluid communication with the laparoscopic field; and
automatically applying suction from the vacuum source to the laparoscopic field when the pressure monitored reaches a set-point, whereupon smoke within the laparoscopic field is evacuated from said laparoscopic field via said second instrument port and said vacuum source.

25. The method of claim 24 wherein said pneumatically-operated smoke evacuator regulator device continuously monitors the pressure of said insufflation gas in said laparoscopic field via said first instrument port and wherein said method additionally comprises:
coupling said pneumatically-operated smoke evacuator regulator device to a second instrument port in fluid communication with said laparoscopic field;
establishing said set-point for a desired pressure of said gas within said laparoscopic field; and
operating said pneumatically-operated smoke evacuator regulator device to monitor the pressure of said gas in said laparoscopic field and automatically coupling said second instrument port to said vacuum source when said pressure of said gas monitored in said laparoscopic field reaches said set-point, whereupon smoke within said laparoscopic field is evacuated from said laparoscopic field via said second instrument port and said vacuum source.

26. The method of claim 25, wherein said laparoscopic field is confined and wherein said method additionally comprising:
providing a smoke probe including said second instrument port
extending said smoke probe into said confined laparoscopic field, said smoke probe additionally comprising a valve and a thin elongated tubular member having a longitudinally extending passageway extending therethrough and terminating at an open distal end, said valve being interposed between said second instrument port and said longitudinally extending passageway; and
disposing said open distal end closely adjacent a source of smoke within said confined laparoscopic field and opening said valve, whereupon smoke produced by the source of smoke is evacuated from the confined laparoscopic field via said smoke probe.

27. The method of claim 26, wherein said first instrument port comprises a portion of a gel port configured for location within an opening in the body of the patient in communication with the laparoscopic field, said gel port including a penetrable member, and wherein said method comprises penetrating said penetrable member by said thin elongated tubular member of said smoke probe.
